(19) United States
(12) Patent Application Publication
(10) Pub. No.: US 2022/0084203 A1
(43) Pub. Date: Mar. 17, 2022

(54) SYSTEM AND METHODS FOR INTERACTIVE LESION CHARACTERIZATION

(71) Applicant: Delphinus Medical Technologies, Inc., Novi, MI (US)

(72) Inventors: Olivier Roy, Novi, MI (US); Mark Forchette, Novi, MI (US); Bruno Dacquay, Novi, MI (US)

(21) Appl. No.: 17/537,396

(22) Filed: Nov. 29, 2021

Related U.S. Application Data
(63) Continuation of application No. PCT/US2020/035325, filed on May 29, 2020.
(60) Provisional application No. 62/855,387, filed on May 31, 2019.
(60) Provisional application No. 62/963,940, filed on Jan. 21, 2020.

(51) Int. Cl. G06T 7/00; G06V 10/84

(52) U.S. Cl. CPC G06T 7/0012; G06V 10/84; G06V 2201/03; G06T 2207/20076; G06T 2207/30096

(57) ABSTRACT

Described herein are systems, and methods for aiding a user to classify a volume of tissue. A system as described herein may comprise: a plurality of parameters associated with image characteristics related to one or more images of a volume of tissue; a plurality of probabilities each associated with a potential classification of a region of the volume of tissue and each related to one or more parameters of the plurality of parameters, wherein the plurality of probabilities are assumed to be independent of one another; and a graphical display visible to a user, the display comprising a graphical representation of a subset of relevant parameters of the plurality of parameters, wherein the graphical representation informs a classification of the region of the volume of tissue, and wherein a probability of the classification is represented visually.

### CROSS-REFERENCE

This patent application is a continuation of PCT Application No. PCT/US2020/035325 filed May 29, 2020, which claims the benefit of U.S. Provisional Application Ser. No. 62/855,387, filed May 31, 2019, and U.S. Provisional Application Ser. No. 62/963,940, filed Jan. 21, 2020, each of which is entirely incorporated herein by reference for all purposes.

### BACKGROUND

Breast cancer may be one of the leading causes of cancer mortality among women. Early detection of breast disease can lead to a reduction in the mortality rate. However, problems exist with the sensitivity and specificity of current standards for breast cancer screening. These problems are substantial within the subset of young women with dense breast tissue who are at an increased risk for cancer development.

Some imaging modalities such as ultrasound imaging may be advantageous to others in early detection of some masses such as breast tumors. However, the degree of skill needed to interpret the ultrasound images may lead to high number of false positives which may lead to additional imaging and biopsies.

There are needs for improved systems to train healthcare professionals to make informed decisions, particularly for interpreting ultrasound images of breasts.

### SUMMARY

According to some aspects of the disclosure, a system for aiding a user to classify a volume of tissue is provided. The system for aiding a user to classify a volume of tissue may comprise a plurality of parameters associated with image characteristics related to one or more images of a volume of tissue, a plurality of probabilities each associated with a potential classification of a region of the volume of tissue, and each related to one or more parameters of the plurality of parameters. In some embodiments, the plurality of probabilities may be assumed to be independent of one another. In some embodiments, the system may further comprise a graphical display visible to a user, the display comprising a graphical representation of a subset of relevant parameters of the plurality of parameters. In some embodiments, the graphical representation may inform a classification of the region of the volume of tissue, and wherein a probability of the classification may be represented visually.

In some embodiments, the graphical representation may comprise a matrix style display wherein rows or columns of the matrix comprise all or a subset of the plurality of potential classifications of the image and wherein columns or rows comprise the subset of relevant parameters of the plurality of parameters, and wherein an element of the matrix provides a visible representation of a probability of a potential classification associated with a parameter of the subset of relevant parameters. In some embodiments, the system for aiding a user to classify a volume of tissue may further comprise a parameter selection panel. In some embodiments, the parameter selection panel may comprise all or a subset of plurality of parameters. In some embodiments, the parameter selection panel may be visible of a user interface of an electronic device such as a tablet or a smart phone. In some embodiments, a probability of the potential classification may be displayed using a score value or a color saturation or grey scale variation or a size variation of a visual marker. In some embodiments, a probability associated with the potential classification is a conditional probability. In some embodiments, the conditional probability is computed using Bayes theorem. In some embodiments, the image comprises one or more acoustic renderings of the volume of tissue, the one or more acoustic renderings comprising a tissue characteristics related to sound propagation through the volume of tissue. In some embodiments, at least one of the acoustic renderings may comprise at least a transmission image and a reflection image. In some embodiments, the image may be a combined or a derived image. In some embodiments, the combined image may comprise a plurality of reflection images or transmission images. In some embodiments, the combined mage may comprise at least one reflection image and one transmission image. In some embodiments, a transmission image may comprise a sound speed image or an attenuation image. In some embodiments, the region of the volume of tissue may comprise a tissue lesion. In some embodiments, the classification of lesion may comprise a cancer, a fibroadenoma, a cyst, a nonspecific benign mass, or an unidentifiable mass. In some embodiments of the system for aiding a user to classify a volume of tissue, the rows or the columns of the matrix may represent the lesion type. In some embodiments, at least one image of the system for aiding a user to classify a volume of tissue may comprise at least one image selected from the group consisting of an enhanced reflection image, a B-mode reflection image, a sound speed image, and a stiffness representation. In some embodiments, the plurality of parameters may comprise at least one morphological feature or at least one qualitative parameter. In some cases, the plurality of parameters may comprise at least one quantitative parameter or a volumetric parameter. In some embodiments, the plurality of parameters may comprise at least one parameter derived from a plurality of image positions along an anterior-posterior axis of the tissue. In some embodiments, at least one of the parameters may comprise a comparison of a region margin or a region shape. In some embodiments, the plurality of parameters may comprise at least one parameter derived from two or more images selected from the group consisting of an enhanced reflection image, a B-mode reflection image, a sound speed image, and a stiffness representation. At least one of said parameters may comprise a comparison of a region margin or a region shape. In some embodiments, the graphical representation provides classification guidance. In some embodiments, the system for aiding a user to classify a volume of tissue does not provide a classification to the user.

According to some aspects of the present disclosure, a method for aiding a user to classify a volume of tissue is disclosed. The classification method may comprise receiving a set of parameters associated with image characteristics related to at least one image of the volume of tissue, providing a set of probabilities associated with a potential classification of a region of the volume of tissue and each related to at least one parameter of the set of parameters. The set of probabilities may be assumed to be independent of one another. The method may further comprise graphically representing the set of probabilities on a display visible to a user. A graphical representation on the display may indicate a subset of relevant parameters of the plurality of parameters to the user, wherein the graphical representation may inform a classification of the region of the volume of tissue, and wherein a probability of the classification may be represented visually.

In some embodiments, the method may further comprise, selecting one or more parameters from a parameter selection panel, wherein the parameter selection panel comprises all or a subset of the plurality of parameters. In some embodiments, the graphical representation may comprise a matrix style display wherein rows or columns of the matrix may comprise all or a subset of the plurality of potential classifications of the image and wherein columns or rows may comprise the subset of relevant parameters of the plurality of parameters, and wherein an element of the matrix may provide a visible representation of a probability of a potential classification associated with a parameter of the subset of relevant parameters. In some embodiments, the parameter selection panel may be visible on a user interface of an electronic device. The electronic device may be a table or a smartphone. In some embodiments, the method may comprise displaying a score value associated with a probability of the potential classification. In some embodiments, the method may comprise varying a color saturation or grey scale. In some embodiments, the method may comprise varying a size of a visual marker. In some embodiments, a probability associated with the potential classification is a conditional probability. In some embodiments, the conditional probability is computed using Bayes theorem. In some embodiments, the image comprises one or more acoustic renderings of the volume of tissue, the one or more acoustic renderings comprising a tissue characteristics related to sound propagation through the volume of tissue. In some embodiments, at least one of the acoustic renderings may comprise at least a transmission image and a reflection image. In some embodiments, the image may be a combined or a derived image. In some embodiments, the combined image may comprise a plurality of reflection images or transmission images. In some embodiments, the combined mage may comprise at least one reflection image and one transmission image. In some embodiments, a transmission image may comprise a sound speed image or an attenuation image. In some embodiments, the region of the volume of tissue may comprise a tissue lesion. In some embodiments, the classification of lesion may comprise a cancer, a fibroadenoma, a cyst, a nonspecific benign mass, or an unidentifiable mass. In some embodiments of the system for aiding a user to classify a volume of tissue, the rows or the columns of the matrix may represent the lesion type. In some embodiments, at least one image of the system for aiding a user to classify a volume of tissue may comprise at least one image selected from the group consisting of an enhanced reflection image, a B-mode reflection image, a sound speed image, and a stiffness representation. In some embodiments, the plurality of parameters may comprise at least one morphological feature or at least one qualitative parameter. In some embodiments, the plurality of parameters may comprise at least one quantitative parameter or a volumetric parameter. In some embodiments, the plurality of parameters may comprise at least one parameter derived from a plurality of image positions along an anterior-posterior axis of the tissue. In some embodiments, at least one of the parameters may comprise a comparison of a region margin or a region shape. In some embodiments, the plurality of parameters may comprise at least one parameter derived from two or more images selected from the group consisting of an enhanced reflection image, a B-mode reflection image, a sound speed image, and a stiffness representation. At least one of said parameters may comprise a comparison of a region margin or a region shape. In some embodiments, a method for training a user to classify an image may further be presented. In some embodiments, the method, in addition to the method described above, may comprise receiving a classification from the user, and providing a known classification to the user. In some embodiments, the method further comprises providing classification guidance. In some embodiments, the method does not provide a classification to the user.

According to some aspects of the present disclosure, a method for building a dataset is disclosed. The method, in addition to the method described above, may comprise providing a set of classified image data, extracting a set of parameters of the set of classified image data, wherein the set of parameters are based on a particular image characteristic, calculating a Bayes probability of each parameter of the set of parameters yielding a characterization, storing a set of Bayes probabilities based on the Bayes probability of each parameter of the set of parameters in a database, and performing the method for aiding a user to classify a volume of tissue as described herein. The database may be updated based on the classification of the region of the volume of tissue.

According to some aspects of the present disclosure, a non-transitory computer readable medium is disclosed. The non-transitory computer readable storage medium may comprise machine-executable code that upon execution by a computing system implements the method of any aspect or embodiment disclosed herein.

According to some aspects of the present disclosure, a system for aiding a user to classify a volume of tissue is disclosed. The system may comprise: a computing system comprising a memory, the memory comprising instructions aiding a user to classify a volume of tissue, wherein the computer system is configured to execute instructions to at least: receive a set of parameters associated with image characteristics related to at least one image of the volume of tissue; provide a set of probabilities associated with a potential classification of a region of the volume of tissue and each related to at least one parameter of the set of parameters, wherein the set of probabilities are assumed to be independent of one another; and graphically represent the set of probabilities on a display visible to a user, wherein a graphical representation on the display indicates a subset of relevant parameters of the plurality of parameters to the user, wherein the graphical representation informs a classification of the region of the volume of tissue, and wherein a probability of the classification is represented visually.

According to some aspects of the present disclosure, a system for aiding a user to classify a volume of tissue is disclosed. The system may comprise: a computer memory configured to store (i) a plurality of parameters associated with image characteristics related to one or more images of a volume of tissue; and (ii) a plurality of probabilities each associated with a potential classification of a region of the volume of tissue and each related to one or more parameters of the plurality of parameters, wherein the plurality of probabilities is assumed to be independent of one another; and a graphical display visible to a user, the display comprising a graphical representation of a subset of relevant parameters of the plurality of parameters, wherein the graphical representation informs a classification of the region of the volume of tissue, and wherein a probability of the classification is represented visually.

In some embodiments, the graphical representation comprises a matrix style display wherein rows or columns of the matrix comprise all or a subset of the plurality of potential classifications of the image and wherein columns or rows comprise the subset of relevant parameters of the plurality of parameters, and wherein an element of the matrix provides a visible representation of a probability of a potential classification associated with a parameter of the subset of relevant parameters. In some embodiments, the graphical representation comprises a parameter selection panel, wherein the parameter selection panel comprises all or a subset of the plurality of parameters. In some embodiments, the parameter selection panel is visible on a user interface of an electronic device. In some embodiments, the electronic device is a tablet or smartphone.

In some embodiments, a probability of the potential classification is displayed using a score value. In some embodiments, a probability of the potential classification is displayed using a color saturation or grey scale variation. In some embodiments, a probability of the potential classification is displayed using a size variation of a visual marker. In some embodiments, a probability associated with the potential classification is a conditional probability. In some embodiments, the conditional probability is computed using Bayes theorem.

In some embodiments, the image comprises one or more acoustic renderings of the volume of tissue, the one or more acoustic renderings comprising tissue characteristics related to sound propagation through the volume of tissue. In some embodiments, the one or more acoustic renderings comprises at least a transmission image and a reflection image. In some embodiments, the image comprises a combined or a derived image. In some embodiments, the combined image comprises a plurality of reflection images. In some embodiments, the combined image comprises a plurality of transmission images. In some embodiments, the combined image comprises at least one reflection image and at least one transmission image. In some embodiments, a transmission image comprises a sound speed image or an attenuation image.

In some embodiments, the region of the volume of tissue comprises a tissue lesion. In some embodiments, the classification of the lesion comprises a cancer, a fibroadenoma, a cyst, a nonspecific benign mass, or an unidentifiable mass. In some embodiments, the rows or the columns of the matrix represent the lesion type. In some embodiments, the at least one image comprises at least one image selected from the group consisting of an enhanced reflection image, a B-mode reflection image, a sound speed image, and a stiffness representation.

In some embodiments, the plurality of parameters comprises at least one morphological feature. In some embodiments, the plurality of parameters comprises at least one qualitative parameter. In some embodiments, the plurality of parameters comprises at least one quantitative parameter. In some embodiments, the plurality of parameters comprises at least one volumetric parameter. In some embodiments, the plurality of parameters comprises at least one parameter derived from a plurality of image positions along an anterior-posterior axis of the tissue.

In some embodiments, the at least one parameter derived from the plurality of image positions comprises a comparison of a region margin or a region shape. In some embodiments, the plurality of parameters comprises at least one parameter derived from two or more images selected from the group consisting of an enhanced reflection image, a B-mode reflection image, a sound speed image, and a stiffness representation. In some embodiments, at least one parameter derived from two or more image types comprises a comparison of a region margin or a region shape. In some embodiments, the graphical representation provides classification guidance. In some embodiments, the system for aiding a user to classify a volume of tissue does not provide a classification to the user.

According to some aspects of the present disclosure, a computer-implemented method for aiding a user to classify an image of a volume of tissue is disclosed. The method may comprise: receiving at a processor a set of parameters associated with image characteristics related to at least one image of the volume of tissue; providing from the processor a set of probabilities associated with a potential classification of a region of the volume of tissue and each related to at least one parameter of the set of parameters, wherein the set of probabilities are assumed to be independent of one another; and using the processor to provide a graphical representation of the set of probabilities on a display visible to a user, wherein the graphical representation on the display comprises and indication of a subset of relevant parameters of the plurality of parameters to the user, wherein the graphical representation is configured to inform a classification of the region of the volume of tissue, and wherein a probability of the classification is represented visually.

In some embodiments, the graphical representation comprises a matrix style display wherein rows or columns of the matrix comprises all or a subset of the plurality of potential classifications of the image and wherein columns or rows comprise the subset of relevant parameters of the plurality of parameters, and wherein an element of the matrix provides a visible representation of a probability of a potential classification associated with a parameter of the subset of relevant parameters. In some embodiments, the method further comprises selecting one or more parameters from a parameter selection panel, wherein the parameter selection panel comprises all or a subset of the plurality of parameters. In some embodiments, the parameter selection panel is visible on a user interface of an electronic device. In some embodiments, the electronic device is a tablet or smartphone.

In some embodiments, the method further comprises displaying a score value associated with a probability of the potential classification. In some embodiments, the method further comprises varying a color saturation or grey scale. In some embodiments, the method further comprises varying a size of a visual marker. In some embodiments, a probability associated with the potential classification is a conditional probability. In some embodiments, the conditional probability is computed using Bayes theorem.

In some embodiments, the image comprises one or more acoustic renderings of the volume of tissue, the one or more acoustic renderings comprising a tissue characteristic related to sound propagation through the volume of tissue. In some embodiments, the one or more acoustic renderings comprises at least a transmission image and a reflection image. In some embodiments, the method further comprises combining two or more images to form a combined or a derived image. In some embodiments, the combined image comprises a plurality of reflection images. In some embodiments, the combined image comprises a plurality of transmission images. In some embodiments, the combined image comprises at least one reflection image and at least one transmission image. In some embodiments, a transmission image comprises a sound speed image or an attenuation image.

In some embodiments, the region of the volume of tissue comprises a tissue lesion. In some embodiments, the classification of the lesion comprises a cancer, a fibroadenoma, a cyst, a nonspecific benign mass, or an unidentifiable mass. In some embodiments, the rows or the columns of the matrix represent the lesion type. In some embodiments, the at least one image comprises at least one image selected from the group consisting of an enhanced reflection image, a B-mode reflection image, a sound speed image, and a stiffness representation.

In some embodiments, the plurality of parameters comprises at least one morphological feature. In some embodiments, the plurality of parameters comprises at least one qualitative parameter. In some embodiments, the plurality of parameters comprises at least one quantitative parameter. In some embodiments, the plurality of parameters comprises at least one volumetric parameter. In some embodiments, the plurality of parameters comprises at least one parameter derived from a plurality of image positions along an anterior-posterior axis of the tissue. In some embodiments, the at least one parameter derived from the plurality of image positions comprises a comparison of a region margin or a region shape. In some embodiments, the plurality of parameters comprises at least one parameter derived from two or more of images selected from the group consisting of an enhanced reflection image, a B-mode reflection image, a sound speed image, and a stiffness representation. In some embodiments, the at least one parameter derived from two or more image types comprises a comparison of a region margin or a region shape.

In some embodiments, the method further comprises providing classification guidance. In some embodiments, the method does not provide a classification to the user.

According to some aspects of the present disclosure, a computer-implemented method of training a user to classify an image is disclosed. The method may comprise the method of any aspect or embodiment herein and further comprising: receiving a classification from the user; and providing a known classification to the user.

According to some aspects of the present disclosure, a computer-implemented method of building a dataset is disclosed. The method may comprise: providing a set of classified image data; providing a set of parameters of the set of classified image data, wherein the set of parameters are based on a particular image characteristic; calculating a Bayes probability of each parameter of the set of parameters yielding a characterization; and storing a set of Bayes probabilities based on the Bayes probability of each parameter of the set of parameters in a database.

In some embodiments, the method further comprises performing the method for aiding a user to classify a volume of tissue of any aspect or embodiment. In some embodiments, the method further comprises updating the database based on the classification of the region of the volume of tissue.

According to some aspects of the present disclosure, a non-transitory computer readable medium comprising machine-executable code that upon execution by a computing system implements the method of any aspect or embodiment is disclosed.

According to some aspects of the present disclosure, a system for aiding a user to classify a volume of tissue is disclosed. The system may comprise: a computing system comprising a memory, the memory comprising instructions aiding a user to classify a volume of tissue, wherein the instructions when executed by a processor are configured to at least: receive a set of parameters associated with image characteristics related to at least one image of the volume of tissue; provide a set of probabilities associated with a potential classification of a region of the volume of tissue and each related to at least one parameter of the set of parameters, wherein the set of probabilities are assumed to be independent of one another; and graphically represent the set of probabilities on a display visible to a user, wherein a graphical representation on the display indicates a subset of relevant parameters of the plurality of parameters to the user, wherein the graphical representation informs a classification of the region of the volume of tissue, and wherein a probability of the classification is represented visually.

In some embodiments, the graphical representation comprises a matrix style display wherein rows or columns of the matrix comprise all or a subset of the plurality of potential classifications of the image and wherein columns or rows comprise the subset of relevant parameters of the plurality of parameters, and wherein an element of the matrix provides a visible representation of a probability of a potential classification associated with a parameter of the subset of relevant parameters.

In some embodiments, the system comprises a parameter selection panel, wherein the parameter selection panel comprises all or a subset of the plurality of parameters. In some embodiments, the parameter selection panel is visible on a user interface of an electronic device. In some embodiments, the electronic device is a tablet or smartphone.

In some embodiments, a probability of the potential classification is displayed using a score value. In some embodiments, a probability of the potential classification is displayed using a color saturation or grey scale variation. In some embodiments, a probability of the potential classification is displayed using a size variation of a visual marker. In some embodiments, a probability associated with the potential classification is a conditional probability. In some embodiments, the conditional probability is computed using Bayes theorem.

In some embodiments, the image comprises one or more acoustic renderings of the volume of tissue, the one or more acoustic renderings comprising tissue characteristics related to sound propagation through the volume of tissue. In some embodiments, the one or more acoustic renderings comprises at least a transmission image and a reflection image. In some embodiments, the image comprises a combined or a derived image. In some embodiments, the combined image comprises a plurality of reflection images. In some embodiments, the combined image comprises a plurality of transmission images. In some embodiments, the combined image comprises at least one reflection image and at least one transmission image. In some embodiments, a transmission image comprises a sound speed image or an attenuation image.

In some embodiments, the region of the volume of tissue comprises a tissue lesion. In some embodiments, the classification of the lesion comprises a cancer, a fibroadenoma, a cyst, a nonspecific benign mass, or an unidentifiable mass. In some embodiments, the rows or the columns of the matrix represent the lesion type. In some embodiments, the at least one image comprises at least one image selected from the group consisting of an enhanced reflection image, a B-mode reflection image, a sound speed image, and a stiffness representation.

In some embodiments, the plurality of parameters comprises at least one morphological feature. In some embodiments, the plurality of parameters comprises at least one qualitative parameter. In some embodiments, the plurality of parameters comprises at least one quantitative parameter. In some embodiments, the plurality of parameters comprises at least one volumetric parameter. In some embodiments, the plurality of parameters comprises at least one parameter derived from a plurality of image positions along an anterior-posterior axis of the tissue. In some embodiments, the at least one parameter derived from the plurality of image positions comprises a comparison of a region margin or a region shape. In some embodiments, the plurality of parameters comprises at least one parameter derived from two or more images selected from the group consisting of an enhanced reflection image, a B-mode reflection image, a sound speed image, and a stiffness representation. In some embodiments, the at least one parameter derived from two or more image types comprises a comparison of a region margin or a region shape.

In some embodiments, the graphical representation provides classification guidance. In some embodiments, the system for aiding a user to classify a volume of tissue does not provide a classification to the user.

According to some aspects of the present disclosure, a method of classifying a lesion within a volume of tissue is disclosed. The method may comprise: receiving from an ultrasound transducer at least one reflection rendering comprising sound reflection data within the volume of tissue; identifying a region of interest within the at least one reflection rendering; receiving from the ultrasound transducer at least one combined rendering comprising sound speed data and sound reflection data within the volume of tissue; identifying a second region of interest within the at least one combined rendering; and classifying a lesion within the volume of tissue based on a similarity or lack thereof of the first region of interest and the second region of interest.

In some embodiments, the method further comprises determining a qualitative image parameter based on a similarity or lack thereof of the first region of interest and the second region of interest. In some embodiments, the method further comprises inputting the qualitative image parameter into a classifier model. In some embodiments, the method further comprises inputting the qualitative image parameter into the method for aiding a user to classify the volume of tissue of any of claims aspect or embodiment.

According to some aspects of the present disclosure, A method of classifying a lesion within a volume of tissue is disclosed. The method may comprise: receiving from an ultrasound transducer a first speed rendering at a first anterior-posterior position, the first speed rendering comprising sound speed data within the volume of tissue; identifying a region of interest within the first speed rendering; receiving from the ultrasound transducer a second speed rendering at a second anterior-posterior position, the second speed rendering comprising sound speed data within the volume of tissue; identifying a second region of interest within the second speed rendering; and classifying a lesion within the volume of tissue based on a similarity or lack thereof of the first region of interest and the second region of interest.

In some embodiments, the method further comprises determining a qualitative image parameter based on a similarity or lack thereof of the first region of interest and the second region of interest. In some embodiments, the method further comprises inputting the qualitative image parameter into a classifier model. In some embodiments, the method further comprises inputting the qualitative image parameter into the method for aiding a user to classify the volume of tissue of any aspect or embodiment.

According to some aspects of the present disclosure, A method of classifying a lesion within a volume of tissue is disclosed. The method may comprise: receiving from an ultrasound transducer a first stiffness rendering at a first anterior-posterior position, the first stiffness rendering comprising a combination of sound speed and sound attenuation data within the volume of tissue; identifying a region of interest within the first attenuation rendering; receiving from the ultrasound transducer a second attenuation rendering at a second anterior-posterior position, the second attenuation rendering comprising a second combination of sound speed and sound attenuation within the volume of tissue; identifying a second region of interest within the second attenuation rendering; and classifying a lesion within the volume of tissue based on a similarity or lack thereof of the first region of interest and the second region of interest.

In some embodiments, the method further comprises determining a qualitative image parameter based on a similarity or lack thereof of the first region of interest and the second region of interest. In some embodiments, the method further comprises inputting the qualitative image parameter into a classifier model. In some embodiments, the method further comprises inputting the qualitative image parameter into the method for aiding a user to classify the volume of tissue of any aspect or embodiment.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein) of which:

FIG. 1A illustrates a flow chart of method for aiding a user to classify a volume of tissue, in accordance with some embodiments.

FIG. 1B illustrates a flow chart of a method for building a dataset, in accordance with some embodiments.

FIG. 1C illustrates a flow chart of a method for training a user to classify an image, in accordance with some embodiments.

FIG. 2 illustrates a graphical display for a system for aiding a user to characterize images, in accordance with some embodiments.

FIG. 3A, FIG. 3B, FIG. 3C, FIG. 3D, and FIG. 3E illustrate examples of sound speed (FIG. 3A), sound attenuation (FIG. 3B), sound reflection (FIG. 3C), wafer (FIG. 3D), and stiffness (FIG. 3E) ultrasound tomography (UT) images of a breast, respectively.

FIG. 4 illustrates examples of tissue boundaries and shapes for UT images of breast tissue.

FIG. 5 illustrates examples of distortion and spiculation in different rendering of ultrasonic images.

FIG. 6A and FIG. 6B illustrate examples of a flow parameter in the sound speed images.

FIG. 7 illustrates example of a persist parameter in the reflection and wafer images.

FIG. 8A illustrates an example of the range of grayscale shades applied to wafer, sound speed, and reflection images.

FIG. 8B illustrates an example of a range of colorscale on a stiffness image.

FIG. 9 illustrates an example of persistence of color or lack thereof in stiffness images.

FIG. 10 illustrates an example of existence of lucent halo or lack thereof in sound speed images according to some embodiments.

FIG. 11A, FIG. 11B, and FIG. 11C illustrate various examples of parameters related to large cancers in wafer, reflection, and stiffness UT images, respectively.

FIG. 12A, FIG. 12B, and FIG. 12C illustrate various examples of parameters related to small cancers in wafer, reflection, and stiffness UT images, respectively.

FIG. 13A, FIG. 13B, FIG. 13C, and FIG. 13D illustrate examples of soft dense tissue and stiff dense tissue on different image types according to some embodiments in wafer, reflection, sound speed, and stiffness UT images, respectively.

FIG. 14A, FIG. 14B, FIG. 14C, and FIG. 14D illustrate examples of fatty lobule on different image types according to some embodiments in wafer, reflection, sound speed, and stiffness UT images, respectively.

FIG. 15A, FIG. 15B, FIG. 15C, and FIG. 15D illustrate examples of cyst on different image types according to some embodiments in wafer, reflection, sound speed, and stiffness UT images, respectively.

FIG. 16A, FIG. 16B, FIG. 16C, and FIG. 16D illustrate examples of small cyst on different image types according to some embodiments in wafer, reflection, sound speed, and stiffness UT images, respectively.

FIG. 17A, FIG. 17B, FIG. 17C, and FIG. 17D illustrate examples of soft fibroadenoma on different image types according to some embodiments. in wafer, reflection, sound speed, and stiffness UT images, respectively

FIG. 18A, FIG. 18B, FIG. 18C, and FIG. 18D illustrate examples of stiff fibroadenoma on different image types according to some embodiments.

FIG. 19A, FIG. 19B, FIG. 19C, and FIG. 19D illustrate examples of general appearance of cancers on different ultrasound image types according to some embodiments in wafer, reflection, sound speed, and stiffness UT images, respectively.

FIG. 20A, FIG. 20B, and FIG. 20C illustrate examples of appearance of large cancers on different ultrasound image types according to some embodiments in wafer, reflection, and stiffness UT images, respectively.

FIG. 21A and FIG. 21B illustrate examples of spiculation appearance on sound speed and reflection UT images, respectively.

FIG. 21C and FIG. 21D illustrate examples of cancer margin appearance on sound speed and wafer UT image, respectively

FIG. 22 illustrates a table summary of lesion characteristics related to different ultrasound image types, in accordance with some embodiments.

FIG. 23 illustrates a graphical user interface, in accordance with some embodiments.

FIG. 24 illustrates a schematic of a computer system that is programmed or otherwise configured to implement methods and systems provided herein.

FIG. 25 illustrates an example of a tablet's physical controls with a graphical user interface, in accordance with some embodiments.

FIG. 26 illustrates a schematic of an application provision system comprising one or more databases accessed by a relational database management system, in accordance with some embodiments.

FIG. 27 is an example table of probabilities for associated parameter values and characterizations.

### DETAILED DESCRIPTION

The methods and systems described herein provide training tools for healthcare professionals to enable them to make more accurate classifications on images of volumes of tissue. The methods and systems disclosed herein, may lead a user, for example, a physician to distinguish between different possible classifications of images of a volume of tissue by providing the probability of various classifications of an image for parameters that may be common between different classifications. The training tool provided herein may better facilitate physicians in detecting the early sings of disease and reducing false positive detections by informing the physicians of likelihood of various classifications on an image of a volume of tissue.

Although various examples of the present disclosure may comprise ultrasound images of breast tissue, the methods and systems described herein can be implemented for any other imaging modality such as magnetic resonance imaging (MRI) or computed tomography (CT) or positron emission tomography (PET) or a combination of imaging modalities. Devices and methods of use as disclosed herein may be used to characterize a number of biological tissues to provide a variety of diagnostic information. A biological tissue may comprise an organ or tissue of a patient or subject. The methods and systems described herein can further be implemented on the images from any organ or tissue of the body. The organ or tissue may comprise for example: a muscle, a tendon, a ligament, a mouth, a tongue, a pharynx, an esophagus, a stomach, an intestine, an anus, a liver, a gallbladder, a pancreas, a nose, a larynx, a trachea, lungs, a kidneys, a bladder, a urethra, a uterus, a vagina, an ovary, a testicle, a prostate, a heart, an artery, a vein, a spleen, a gland, a brain, a spinal cord, a nerve, etc, to name a few.

Other biological tissue may comprise a body part, such as a brain, a foot, a hand, a knee, an ankle, an abdomen, muscles, a tendon, a ligament, a mouth, a tongue, a pharynx, an esophagus, a stomach, an intestine, an anus, a liver, a gallbladder, a pancreas, a nose, a larynx, a trachea, lungs, a kidney, a bladder, a urethra, a uterus, a vagina, an ovary, a breast, a testes, a prostate, a heart, an artery, a vein, a spleen, a gland, a spinal cord, a nerve, or any other body part. A body part may be operatively attached to or contained within a living human being. In some embodiments, the body part comprises muscular tissue, fatty tissue, bone, etc. The body part may be a human body part. The body part may be a body part of a non-human animal, such as a body part of a mouse, cat, dog, bird, pig, sheep, bovine, horse, or non-human primate.

The terminology used herein is for the purpose of describing particular embodiments only and is not intended to be limiting of the claims. As used in the description of the embodiments and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” is optionally construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” is optionally construed to mean “upon determining” or “in response to determining” or “in accordance with a determination” or “upon detecting” or “in response to detecting” that the stated condition precedent is true, depending on the context.

As used herein, and unless otherwise specified, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, 0.1%, or 0.05% of a given value or range.

As used herein, the terms “comprises,” “comprising,” or any other variation thereof, are intended to cover a nonexclusive inclusion, such that a process, method, article, or apparatus that comprises a list of elements does not include only those elements but may include other elements not expressly listed or inherent to such process, method, article, or apparatus.

As used herein, the terms “subject” and “patient” are used interchangeably. As used herein, the terms “subject” and “subjects” refers to an animal (e.g., birds, reptiles, and mammals), a mammal including a primate (e.g., a monkey, chimpanzee, and a human) and a non-primate (e.g., a camel, donkey, zebra, cow, pig, horse, cat, dog, rat, and mouse). In certain embodiments, the mammal is 0 to 6 months old, 6 to 12 months old, 1 to 5 years old, 5 to 10 years old, 10 to 15 years old, 15 to 20 years old, 20 to 25 years old, 25 to 30 years old, 30 to 35 years old, 35 to 40 years old, 40 to 45 years old, 45 to 50 years old, 50 to 55 years old, 55 to 60 years old, 60 to 65 years old, 65 to 70 years old, 70 to 75 years old, 75 to 80 years old, 80 to 85 years old, 85 to 90 years old, 90 to 95 years old or 95 to 100.

As used herein, the term “user” refers to a healthcare professional or any individual using the methods and systems of the present disclosure including but not limited to physicians such as radiologists.

Unless otherwise defined, all technical terms used herein have the same meaning as commonly understood by one of ordinary skill in the art to which this invention belongs.

Reference will now be made in detail to various embodiments, examples of which are illustrated in the accompanying drawings. In the following detailed description, numerous specific details are set forth in order to provide a thorough understanding of the present disclosure and the described embodiments. However, the embodiments of the present disclosure are optionally practiced without these specific details. In other instances, well-known methods, procedures, components, and circuits have not been described in detail so as not to unnecessarily obscure aspects of the embodiments. In the drawings, like reference numbers designate like or similar steps or components.

### Graphical Display

Systems and methods for aiding a user to classify a volume of tissue of the present disclosure may comprise a visual aid to help train and assist users in recognizing lesion types in medical images. As an alternative or addition to presenting a classification to user, the systems and methods for aiding a user to classify a volume of tissue disclosed herein may facilitate a user's determination of a classification of a volume of tissue. The systems and methods for aiding a user to classify a volume of tissue disclosed herein may be used as a training tool for the user. For example, a system may bring one or more characteristics or parameters of a volume of tissue to the attention of a user. A system may visually represent which image characteristics or parameters may be important to consider when determining a classification. A system may visually represent a likelihood of one or more potential classifications of a volume of tissue to a user. A visual representation of a “likelihood,” “importance,” etc. may be related to a probability of a classification based on one or more parameters. A system for aiding a user to classify a volume of tissue may have an input output design. The user may enter inputs based on observations of image characteristics of one or a plurality of images. In some cases, the user may enter inputs based on a plurality of raw ultrasound tomography images. These inputs may comprise or may be used to generate image parameters. A system may then analyze the inputs and display the outputs in the form of text or colored signs or other visual presentations.

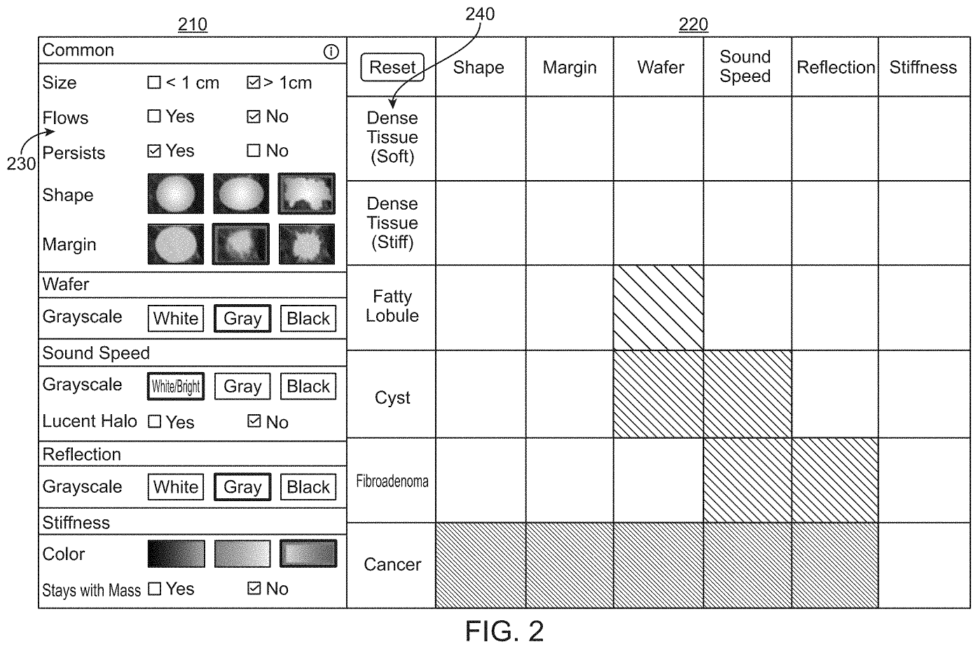

FIG. 2 illustrates a graphical display for a system for aiding a user to characterize images, in accordance with some embodiments. As shown in FIG. 2, a system may comprise a plurality of parameters 230 associated with image characteristics related to one or more images of a volume of tissue. All or a subset of the plurality of parameters may be visualized in a panel on the graphical display hereinafter referred to as characteristics selection panel (CSP) 210 or parameter selection panel. The various characterizations 240 of a tissue and/or mass volume may be visualized on a separate panel on the graphical display hereinafter referred to as characteristics matching panel (CMP) 220.

A system for aiding a user to classify a volume of tissue disclosed herein, may comprise a plurality of probabilities each associated with a potential classification of a region of the volume of tissue. The classifications may comprise particular aspects of the type of tissue, such as to determine whether a mass in the tissue may be a tumor, cyst, fibroadenoma, or other kind of mass. A system for aiding a user to classify a volume of tissue disclosed herein may be used to characterize the tissue to facilitate diagnoses of cancer, assess its type, and determine its extent (e.g., to determine whether a mass in the tissue may be surgically removable), or to assess risk of cancer development (e.g., measuring breast tissue density). The image classification may be related to one or more parameters of the plurality of parameters, described herein. The plurality of parameters may be assumed to be independent of one another. The parameters may be associated with one or more images (for example, ultrasound tomography images) as described herein, for example, with respect to the section “Images” and in the incorporated references.

As shown in FIG. 2, the graphical display may comprise a parameter selection panel 210. The parameter selection may comprise the plurality of parameters 230. The plurality of parameters may be subset of a plurality of parameters associated with an image. For a particular parameter, a qualitative or quantitative value associated that parameter may be shown. A value associated with the parameter may be generated by a user, may be facilitated by a digital processing device, or may be generated by a digital processing device. Various parameters which may be included in the parameter selection panel are described herein, for example, with respect to the section “Image Parameters” and in the incorporated references.

As shown in FIG. 2, the graphical display may comprise a characteristics matching panel 220. The CMP may comprise a visual representation of a plurality of probabilities associated with one or more classifications of the volume of tissue. The visual representation may be a graphical representation. The graphical representation of a system for aiding a user to classify a volume of tissue may comprise a matrix style display. The rows or columns of the matrix may comprise all or a subset of the plurality of potential classifications 240 of the image. The columns or rows may comprise the subset of relevant parameters of the plurality of parameters. An element of the matrix, hereinafter referred to as “cell”, may provide a visible representation of a probability of a potential classification associated with the parameter of the subset of relevant parameters. The parameter selection panel and/or characteristics matching panel may be visible to the user on a user interface of an electronic device such as but not limited to a tablet or a smart phone. Various potential classifications which may be included in the characteristics matching panel are described herein, for example, with respect to the section “Potential Classifications” and in the incorporated references.

The graphical display may visually represent a plurality of probabilities associated with one or more classifications of the volume of tissue. For example, a visual representation of a probability may be represented as a score, a symbol, a color saturation, a grey-scale variation, a size variation of a visual marker, etc. The plurality of probabilities may be generated by a probability engine. The probability engine may be a reverse conditional probability. The probability engine may comprise a Bayes classifier. The probability engine may comprise a decision tree. A probability engine is described herein, for example, with respect to the section “Probability Engine.”

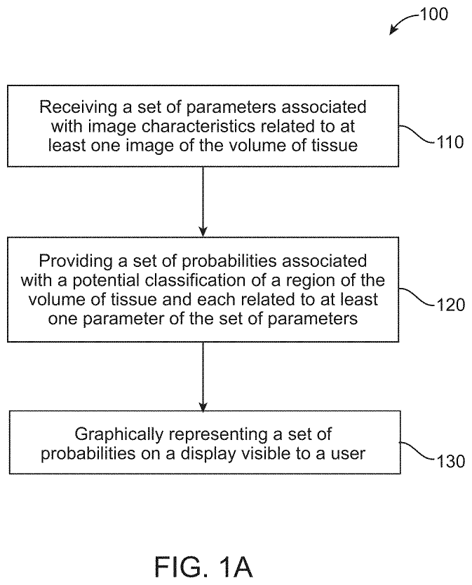

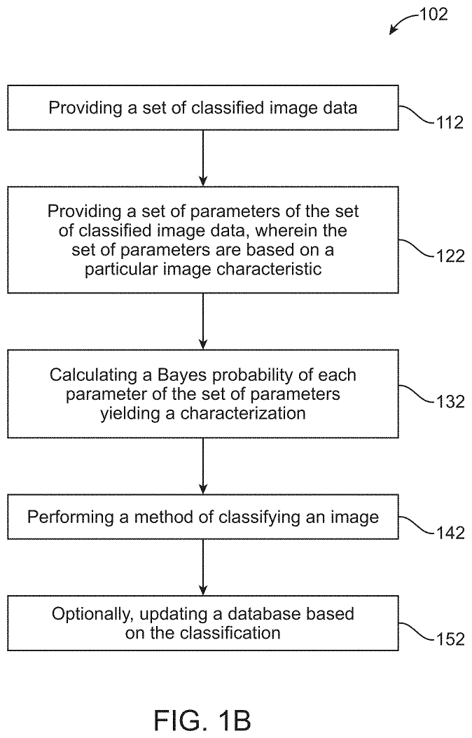

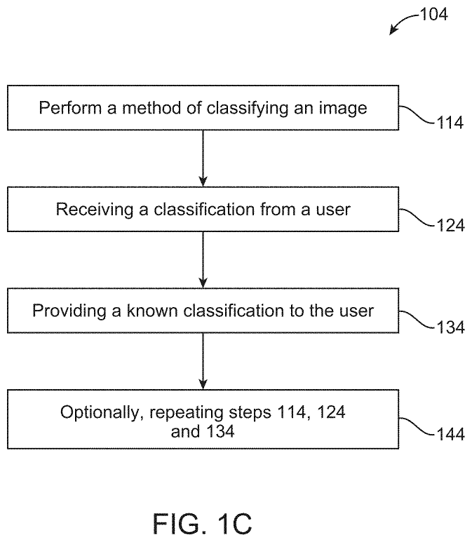

Also disclosed herein are methods for aiding a user to classify a volume of tissue. FIG. 1A shows an example of a method 100 for aiding a user to classify a volume of tissue. In a step 110, a set of parameters associated with image characteristics related to at least one image of the volume of tissue may be received. A set of parameters associated with image characteristics related to at least one image of the volume of tissue may be selected by a user on a graphical user interface. The images may be taken on an imaging system which may be local to a system for aiding a user to classify a volume of tissue as described herein. A user, for example, a medical professional may make an observation and may indicate an image characteristic on a user interface, such as for example on the CSP as described herein above. One or more parameters may be generated from the image characteristic. The one or more parameters may comprise any parameter or combination of parameters as described herein, for example, with respect to the section “Image Parameters” and in the incorporated references.

In a step 120, a set of probabilities associated with a potential classification of a region of the volume of tissue may be provided. Each probability may be related to at least one parameter of the set of parameters. In some cases, one or more of the set of probabilities are assumed to be independent of one another. The parameters may be provided in response to one or more image processing and/or analysis steps at a processor. The processing steps may comprise extraction of one or more parameters from the one or more images. The extraction may use a user selected, computer selected, or computer aided selection of a region of interest (ROI). One or more parameters associated with image characteristics may be extracted from the set of images. The one or more parameters may comprise any parameter or combination of parameters as described herein. Probabilities related to the set of parameters or a subset of parameters associated with potential classifications of a region of tissue volume may be calculated. Each probability may be related to at least one parameter of the set of parameters. The set of probabilities may further be assumed to be independent of one another. In some cases, probabilities may relate to multiple parameters. The probabilities may be determined based on a set of images which may include a database of previously scanned images and/or images of the volume of tissue.

In a step 130, the set of probabilities may be graphically represented on a display visible to a user. A graphical representation on the display may indicate a subset of relevant parameters of the plurality of parameters to the user. The graphical representation may inform a classification of the region of the volume of tissue. A probability of the classification may be represented visually.

Also disclosed herein are methods for building a dataset. FIG. 1B shows an example of a method 102 for building a dataset. At a step 112, the method may comprise providing a set of classified image data. At a step 122, the method may comprise providing a set of parameters of the set of classified image data. The set of parameters may be based on a particular image characteristic. At a step 134, the method may comprise calculating a Bayes probability of each parameter of the set of parameters yielding a characterization. The method may comprise storing a set of Bayes probabilities based on the Bayes probability of each parameter of the set of parameters in a database. At a step 144, the method may comprise performing a method for aiding a user to classify a volume of tissue as described herein. The database may be updated based on the classification of the region of the volume of tissue. In some instances, the dataset may be updated with data from each new patient. The dataset may be updated every scan, every patient, every day, every week, every month, every quarter, every year, etc. The updated dataset may be used to calculate an updated set of probabilities, which may be used in the systems and methods disclosed herein.

Also disclosed herein are methods for training a user to classify an image. FIG. 1C shows an example of a method 104 for training a user to classify an image. At a step 114, the method may comprise receiving a classification from the user. At a step 124, the method may comprise providing a known classification to the user. At a step 134, the method may optionally comprise repeating any of steps 114, 124, and 134 any number of times.

Although the above operations show examples of methods 100, 102, and 104, in accordance with some embodiments, a person of ordinary skill in the art will recognize many variations based on the teachings described herein. The steps may be completed in any order. Steps may be added or deleted. Some of the steps may comprise sub-steps. Many of the steps may be repeated as often as beneficial to the characterization of a target.

One or more steps of the methods 100, 102, and 104 be performed with the circuitry as described herein, for example, one or more of the digital processing device or processor or logic circuitry such as the programmable array logic for a field programmable gate array. The circuitry may be programmed to provide one or more steps of the methods 100, 102, and 104, and the program may comprise program instructions stored on a computer readable memory or programmed steps of the logic circuitry such as the programmable array logic or the field programmable gate array, for example.

### Images

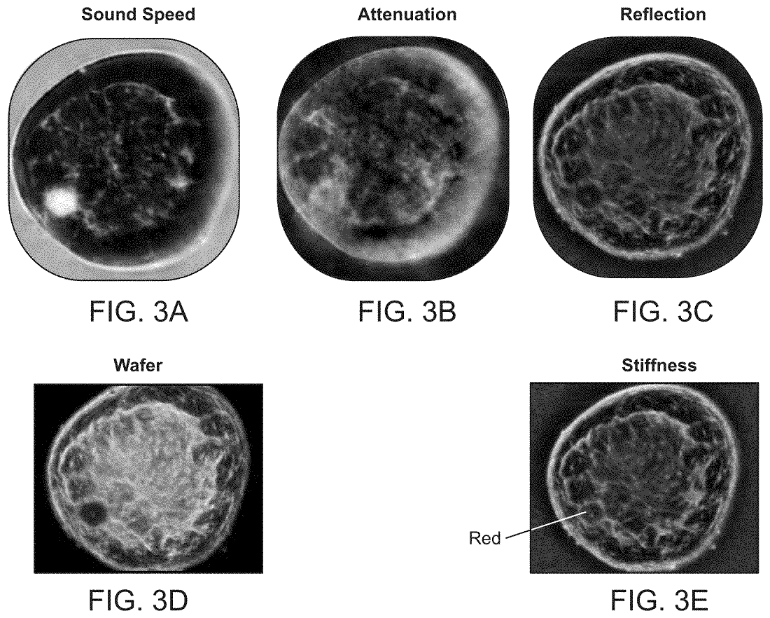

FIG. 3A, FIG. 3B, FIG. 3C, FIG. 3D, and FIG. 3E illustrate examples of sound speed (FIG. 3A), sound attenuation (FIG. 3B), sound reflection (FIG. 3C), wafer (FIG. 3D), and stiffness (FIG. 3E) ultrasound tomography (UT) images of a breast, respectively. The reflection information may be combined with the transmission data (for example, sound speed) to create enhanced reflection images. An example enhanced reflection image is shown in FIG. 3D. In some cases, sound speed data and sound attenuation data may be combined to obtain stiffness images as shown in FIG. 3E. In a stiffness image herein, black/blue represents lower stiffness and orange/red represents higher stiffness. Any number of images or renderings may be used as inputs to a system for aiding a user to classify a volume of tissue as disclosed herein.

In some embodiments, ultrasound images, for example, ultrasound tomography images, are used in the methods and systems of the present disclosure. In such cases, both transmission and reflection information may be used. The transmitted portion of an ultrasound signal may contain information about the sound speed and attenuation properties of the insonified medium. Sound reflection, attenuation, and speed may aid in the differentiation of fat, fibroglandular tissues, benign masses, and malignant cancer.

In some embodiments, systems and methods of the present disclosure may be used in combination with one or more images or renderings that may be used to detect abnormalities (e.g., cancerous tissues) in a human or other animal. As such, in one variation, images used in combination with the system may be used to characterize the tissue to facilitate diagnoses of cancer, assess its type, and determine its extent (e.g., to determine whether a mass in the tissue may be surgically removable), or to assess risk of cancer development (e.g., measuring breast tissue density). In yet another embodiment, images used in combination with the system may be used to characterize or investigate particular aspects of the tissue, such as to determine whether a mass in the tissue may be a tumor, cyst, fibroadenoma, or other kind of mass. Characterizing a lesion in a volume of tissue may be implemented, at least in part, by way of an embodiment, variation, or example of the methods in the incorporated references. Characterizing a lesion in response to an image from a transducer system may be implemented using any other suitable method.

Systems and methods of the present disclosure may comprise receiving one or a plurality of images of a volume of tissue. The images may be received by one or more computer processors described herein. The one or a plurality of images may comprise one or more of a reflection image, a speed image, and an attenuation image. In some embodiments, the systems, devices, and methods disclosed herein may comprise receiving a transmission image. A transmission image may comprise one or more of an attenuation image and a sound speed image. In some embodiments, the plurality of images comprises combined images. In some embodiments, a combined image comprises a plurality of reflection images. In some embodiments, a combined image comprises a plurality of transmission images. In some embodiments, a combined image comprises at least one reflection image and at least one transmission image.

Embodiments of systems and methods described herein may be used in combination with a particular type of image. For example, a particular type of image may be received in relationship to a particular acoustomechanical parameter. Images formed from the various image modalities may be merged in whole or in part to form combined image modalities. In some cases, processing of ultrasound data may be performed using the methods described in the methods in the references incorporated herein. Such methods may include generating a waveform sound speed rendering and generating a reflection rendering.

Receiving one or more image modalities may comprise receiving images generated from a step wise scan in an anterior-posterior axis of a volume of tissue. At each step in a scan, one or more transducer elements may transmit acoustic waveforms into the volume of tissue. At each step in a scan, one or more transducer elements may receive acoustic waveforms from the tissue from the volume of tissue. The received waveforms may be converted to acoustic data. The received waveforms may be amplified. The received waveforms may be digitized. The acoustic data may comprise a speed of energy, a reflection of energy, and/or an attenuation of energy. A received waveform may be amplified and subsequently converted to acoustic data by any processor and associated electronics described herein. The received waveform may be amplified and subsequently converted to acoustic data by a processor and associated electronics.

The one or a plurality of images may be generated from a three-dimensional rendering of an acoustomechanical parameter characterizing sound propagation within a volume of tissue. An acoustomechanical parameter may comprise at least one of, for example, sound speed, sound attenuation, and sound reflection. Each rendering may be formed from one or more “stacks” of 2D images corresponding to a series of “slices” of the volume of tissue for each measured acoustomechanical parameter at each step in a scan of the volume of tissue. In some cases, each rendering may be in response to a model of sound propagation within the volume of tissue generated from the plurality of acoustic data received from the volume of tissue.

FIG. 3A shows an example sound speed image, in accordance with some embodiments. The sound speed rendering may comprise a distribution of sound speed values across the region of the volume of tissue. The two-dimensional sound speed renderings may be associated with slices (e.g. coronal slices) through a volume of tissue. An acoustic sound speed rendering may comprise a three-dimensional (3D) acoustic sound speed rendering that is a volumetric representation of the acoustic sound speed of the volume of tissue. The sound speed rendering can characterize a volume of tissue with a distribution of one or more of: fat tissue (e.g., fatty parenchyma, parenchymal fat, subcutaneous fat, etc.), parenchymal tissue, cancerous tissue, abnormal tissue (e.g., fibrocystic tissue, fibroadenomas, etc.), and any other suitable tissue type within the volume of tissue.

The sound speed map may characterize a real part of the complex valued ultrasound impedance of the volume of tissue, the rate of travel of a waveform through the volume of tissue, a ratio of distance of travel through the volume of tissue over time between transmission and detection, or any other suitable acoustic speed parameter. A stack of 2D acoustic sound speed images may be derived from the real portion of the complex-valued impedance of the tissue and may provide anatomical detail of the tissue

The sound speed rendering may be a waveform sound speed image. Such a method may comprise generating an initial sound speed rendering in response to simulated waveforms according to a travel time tomography algorithm. The initial sound speed rendering may be iteratively optimized until ray artifacts are reduced to a pre-determined a threshold for each of a plurality of sound frequency components. The initial method rendering may be iteratively adjusted until the obtained model is good enough as a starting model for the waveform sound speed method to converge to the true model. Such a method may comprise the method described in U.S. application Ser. No. 14/817,470, which is incorporated herein in its entirety by reference.

FIG. 3B shows an example attenuation image, in accordance with some embodiments. The sound attenuation rendering may comprise a distribution of sound attenuation values across the region of the volume of tissue. An acoustic sound attenuation rendering may comprise one or a plurality of two-dimensional (2D) sound attenuation renderings. The two-dimensional sound attenuation renderings may be associated with slices (e.g., coronal slices) through a volume of tissue. An acoustic sound attenuation rendering may comprise a three-dimensional (3D) acoustic sound attenuation rendering that is a volumetric representation of the acoustic sound attenuation of the volume of tissue. The sound attenuation rendering can characterize a volume of tissue with a distribution of one or more of: fat tissue (e.g., fatty parenchyma, parenchymal fat, subcutaneous fat, etc.), parenchymal tissue, cancerous tissue, abnormal tissue (e.g., fibrocystic tissue, fibroadenomas, etc.), and any other suitable tissue type within the volume of tissue. Generating an acoustic attenuation rendering may comprise a method described in the references incorporated herein.

The sound attenuation map may characterize an imaginary part of the complex valued ultrasound impedance of the volume of tissue, the absorption of a waveform by the volume of tissue, or any other suitable acoustic attenuation parameter. A stack of 2D acoustic sound attenuation images may be derived from the imaginary portion of the complex-valued impedance of the tissue and may provide anatomical detail of the tissue.

The sound attenuation rendering may be a waveform sound attenuation image. Such a method may comprise generating an initial sound speed rendering in response to simulated waveforms according to a travel time tomography algorithm. The initial sound speed rendering may be iteratively optimized until ray artifacts are reduced to a pre-determined a threshold for each of a plurality of sound frequency components. The waveform sound speed rendering may be used as a starting point to generate a waveform attenuation rendering. The initial attenuation rendering may be iteratively adjusted until convergence. Such a method may comprise the method described in U.S. patent application Ser. No. 15/909,661, now U.S. Publication No. 2018/0185002, which is incorporated herein in its entirety by reference.

FIG. 3C shows an example reflection image, in accordance with some embodiments. The sound reflection rendering may comprise a distribution of sound reflection values across the region of the volume of tissue. An acoustic sound reflection rendering may comprise one or a plurality of two-dimensional (2D) sound reflection renderings. The two-dimensional sound reflection renderings may be associated with slices (e.g., coronal slices) through a volume of tissue. An acoustic reflection rendering may comprise a three-dimensional (3D) acoustic reflection rendering that is a volumetric representation of the acoustic reflection of the volume of tissue. The sound reflection rendering can characterize a volume of tissue with a distribution of one or more of: fat tissue (e.g., fatty parenchyma, parenchymal fat, subcutaneous fat, etc.), parenchymal tissue, cancerous tissue, abnormal tissue (e.g., fibrocystic tissue, fibroadenomas, etc.), and any other suitable tissue type within the volume of tissue.

The reflection rendering may comprise envelope detected reflection data (ERF), raw radiofrequency reflection signals (e.g., REF image data, “radiofrequency”, or RF data), which can be converted to a flash B-mode ultrasound image, or any suitable ultrasound image. The distribution of acoustic reflection signals may characterize a relationship (e.g., a sum, a difference, a ratio, etc.) between the reflected intensity and the emitted intensity of an acoustic waveform, a change in the acoustic impedance of a volume of tissue, or any other suitable acoustic reflection parameter. A stack of 2D acoustic reflection images may be derived from changes in acoustic impedance of the tissue and may provide echo-texture data and anatomical detail for the tissue.

In some embodiments, the acoustic reflection rendering may comprise a distribution of acoustic reflection signals received from an array of transducer elements transmitting and receiving at a frequency greater than the frequency of the array of transducer elements used to generate a rendering from another acoustic data type including, for example, the sound speed rendering or the attenuation rendering. In other embodiments, the acoustic reflection rendering may comprise a distribution of acoustic reflection signals received from an array of transducer elements transmitting and receiving at a frequency less than the frequency of the array of transducer elements used to generate a rendering from another acoustic data type including, for example, the sound speed rendering or the attenuation rendering. The low frequencies (˜1 MHz) may provide information on specular reflections (down to ˜1 mm); however, imaging at higher frequencies (˜1 to 5 MHz) may be better able to image the sub-mm granularity that provides information on speckle patterns. Therefore, it may be beneficial to generate a particular acoustic rendering at a particular frequency.

The 3D renderings of any type of acoustic data may comprise combined or merged in whole or in part. In one embodiment, a merged rendering may comprise combining 3D renderings of at least two types of image data. In another embodiment, a merged rendering may comprise combining at least a portion of the plurality of 2D images from at least two types of image data. Any suitable formula or algorithm may be used to merge or fuse the various renderings into a single rendering. A combination may comprise an arithmetic operation relating two or more images (e.g., a sum, a difference, a product, a ratio, a convolution, an average, etc.). In some embodiments, the combined image may be an enhanced reflection image, a stiffness image, etc.

FIG. 3D shows an example enhanced reflection image, in accordance with some embodiments. An enhanced reflection image may be a waveform enhanced reflection (WafER) image. The enhanced reflection rendering may comprise a distribution of sound reflection values across the region of the volume of tissue. An enhanced reflection rendering may comprise one or a plurality of two-dimensional (2D) enhanced reflection renderings. The two-dimensional enhanced reflection renderings may be associated with slices (e.g. coronal slices) through a volume of tissue. An enhanced reflection rendering may comprise a three-dimensional (3D) enhanced reflection rendering that is a volumetric representation of the acoustic reflection of the volume of tissue. The enhanced reflection rendering can characterize a volume of tissue with a distribution of one or more of: fat tissue (e.g., fatty parenchyma, parenchymal fat, subcutaneous fat, etc.), parenchymal tissue, cancerous tissue, abnormal tissue (e.g., fibrocystic tissue, fibroadenomas, etc.), and any other suitable tissue type within the volume of tissue.

An example enhanced reflection image, such as a WafER image, may be an example of a combined image. The enhanced reflection image may be generated from a reflection image generated from detection a reflected signal from a volume of tissue and a speed image. The second reflection image may be generated from a gradient of a sound speed image, and the two reflection images may be combined, as described in the incorporated references. An enhanced image may comprise an embodiment, variation, or example of the system and method for generating an enhanced image of a volume of tissue described in commonly assigned applications: U.S. patent application Ser. No. 15/829,748 and P.C.T. App. No. US2017/064350, which are each incorporated herein by reference in their entirety.

FIG. 3E shows an example stiffness rendering, in accordance with some embodiments. The stiffness rendering may comprise a distribution stiffness across the region of the volume of tissue. A stiffness rendering may comprise one or a plurality of two-dimensional (2D) stiffness renderings. The two-dimensional stiffness renderings may be associated with slices (e.g. coronal slices) through a volume of tissue. A stiffness rendering may comprise a three-dimensional (3D) stiffness rendering that is a volumetric representation of the acoustic reflection of the volume of tissue. The stiffness rendering can characterize a volume of tissue with a distribution of one or more of: fat tissue (e.g., fatty parenchyma, parenchymal fat, subcutaneous fat, etc.), parenchymal tissue, cancerous tissue, abnormal tissue (e.g., fibrocystic tissue, fibroadenomas, etc.), and any other suitable tissue type within the volume of tissue.

A stiffness rendering may be an example of a combined image. The stiffness image may be generated from a sound rendering and an attenuation rendering. In some variations, for instance, combination of values of the sound speed map with corresponding values of the acoustic attenuation map can comprise weighting (e.g., weighting of sound speed values of the sound speed rendering, weighting of attenuation values of the attenuation rending) element values prior to combination to form the combined rendering. A stiffness rendering may comprise an embodiment, variation, or example of the system and method for representing a tissue stiffness described in commonly assigned application U.S. patent application Ser. No. 14/703,746, now U.S. Pat. No. 10,143,443, which is incorporated herein by reference in its entirety.

The images may comprise one or more acoustic renderings of the volume of tissue, the one or more acoustic renderings comprising a representation of sound propagation through the volume of tissue. An example ultrasound system capable of being used with systems and methods of the present disclosure is described herein, see for example, the section titled “Ultrasound System.”

### Image Parameters

As described herein, the system for aiding a user to characterize an image comprises a plurality of parameters associated with image characteristics. Some of these parameters may be common in various types of images as shown in the graphical display of FIG. 2 and some of the parameters may be specific to one or a subset of image types. Some parameters may be indicative of the shape or margin or boundary of a lesion or a volume or tissue. Some parameters may be directed to the periphery of a parenchymal pattern (i.e. fat or glandular margin or interface); looking for potential mass's asymmetry, irregularity, architectural distortion, or spiculations; identifying smaller masses; etc. The plurality of parameters may comprise at least one morphological feature and/or one qualitative parameter and/or one quantitative parameter and/or one volumetric parameter. At least one parameter may be a volumetric parameter.

In some embodiments, a set of parameters can comprise one or a plurality of sound propagation metrics characterizing sound propagation within a tissue. In some embodiments, a sound propagation metric(s) characterizes sound propagation interior to a region of interest, and/or exterior to a region of interest. In some cases, the sound propagation metric(s) characterizes at least one of sound speed, sound attenuation, and sound reflection. In some cases, the sound propagation metric(s) comprises one or more of: a sound speed metric, a reflection metric, an attenuation metric, a user defined score, a morphological metric, and a texture metric.

In some embodiments, a set of parameters may be associated with a region of interest (ROI). An ROI may be a two-dimensional ROI. In some cases, an ROI may correspond to a region comprising all or a portion of a tumor. In some cases, the ROI also comprises a peri-tumoral region. An ROI may substantially circumscribe a lesion within a volume of tissue. An ROI may be user selected. In some instances, user selection of an ROI can indicate a starting point, which can be a point or region which may overlap or be in proximity to a tumor or peri-tumor. For example, a user might indicate a ROI as a closed loop, an arc, a circle, a dot, a line, or an arrow.

Parameters may be extracted from a region of a ROI. In some embodiments, parameters may also be extracted from an expanded region known as the peritumoral region surrounding the ROI. Such expanded region can be generated using various methods. An example method may be to add a uniform distance in each direction. Another example method can include finding the radius of the circle with an equivalent area of the ROI. This radius can be expanded by some multiplicative factor and the difference between the original and expanded radius can be added to each direction of the ROI. Likewise, this method can be modified such that there is a lower or upper threshold for the minimum and maximum radius sizes, respectively. Similarly, such methods can be used to shrink the region of the ROI to generate an inner tumoral ROI.

A parameter from the set of parameters may comprise quantitative parameters, qualitative parameters, or semi-quantitative parameters. Quantitative parameters may comprise, for example, a mean, a median, a mode, a standard deviation, and volume-averages thereof of any acoustic data type. A quantitative parameter may be calculated from a combination of data types. For example, a quantitative parameter may comprise a difference of parameters between a region in the interior of the ROI and in the exterior of the ROI. In another example, a quantitative parameter may comprise a difference between regions of interest, layers, classification of layers, etc. A quantitative parameter may comprise a ratio of a parameter with, for example, another parameter, a known biological property, etc. A quantitative parameter may be weighted by a spatial distribution. A quantitative parameter may be calculated from a volume average of an acoustic data type over, for example, a region of interest, a layer, a plurality of layers, a classification of layers, etc.

Qualitative parameters may comprise one or a combination of the shape, the sharpness, the architecture and/or other characteristics of the morphology renderings. The qualitative parameters may characterize any suitable aspect of the biomechanical property renderings. A qualitative parameter may be converted by a user or a computer into a semi-quantitative parameter, such as “1” for an indistinct margin and “2” for a sharp margin of the region of interest in the acoustic reflection rendering. As another example, a qualitative parameter may be converted by a user or a computer to a semi-quantitative parameter such as a value on an integer scale (e.g., 1 to 5) that classifies the degree to which the qualitative aspect is expressed. For instance, margin sharpness of the region of interest in the acoustic reflection rendering may be classified with a reflection index as “1” if it is very sharp, “3” if it is moderately indistinct, or “5” if it is very indistinct.

Qualitative, quantitative, and semi-quantitative parameters may be combined in order to generate other extended parameters. These extended parameters may comprise the existing Breast Imaging Reporting and Data System (BI-RADS), wherein a lesion is characterized on an integer scale from 1 to 5 but may also comprise other extended parameters comprising acoustic data. The parameters disclosed herein may be time dependent. The time dependence of one or a plurality of parameters may comprise a parameter. In some cases, only a portion of a set of parameters may be used at any instance of time.

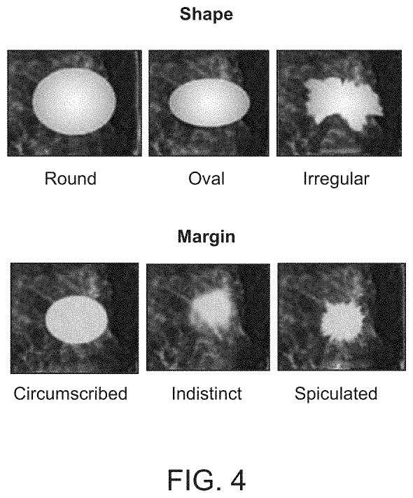

FIG. 4 illustrates examples of tissue boundaries and shapes for UT images of breast tissue. A parameter of a set of parameters of an image or data set may comprise a morphological metric of a region of interest. In some embodiments, the morphological metric comprises at least one of a size, a roundness, an irregularity of a shape, an irregularity of a margin, and a smoothness of a margin. As described above, some parameters associated with image characteristics may include morphological metrics such as a roundness, an irregularity of a shape, an irregularity of a margin, and a smoothness of a margin. Small cancers may exhibit larger architectural distortion. Some ligaments such as Cooper's ligaments may also mimic spiculations as shown in example of FIG. 4. In some cases, it may be easier to find architectural distortions than the mass itself.

The ACR BI-RADS Atlas Ultrasound definition apply for the embodiments of present disclosure if ultrasound images are used. For example, is a lesion is at least two thirds circumscribed, the margin may be considered circumscribed.

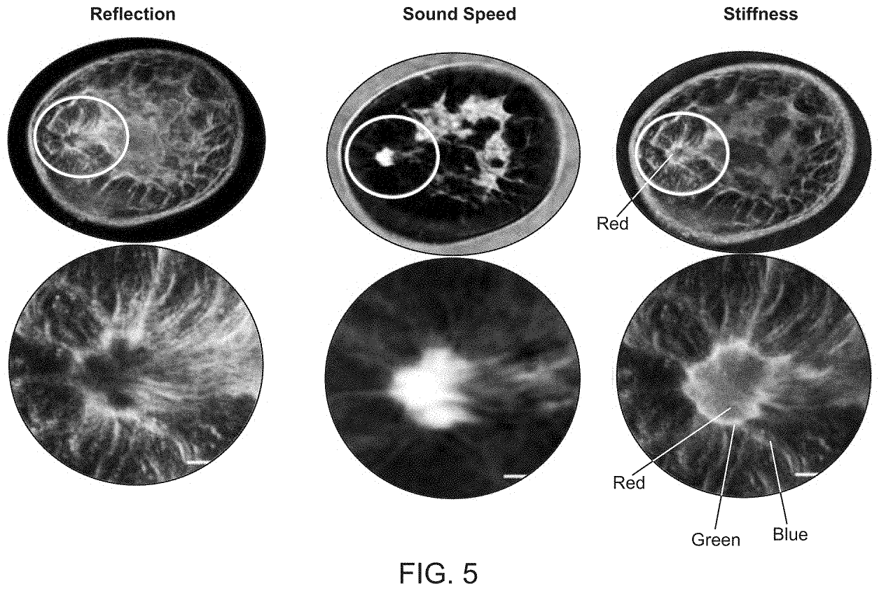

As shown in examples of FIG. 5, distortion may be easier to observer on wafer and reflection images. On the other hand, spiculation may be better observed on sound speed and stiffness images.

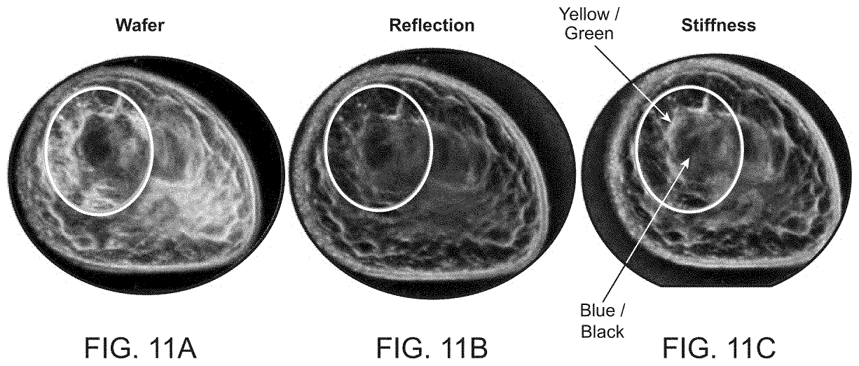

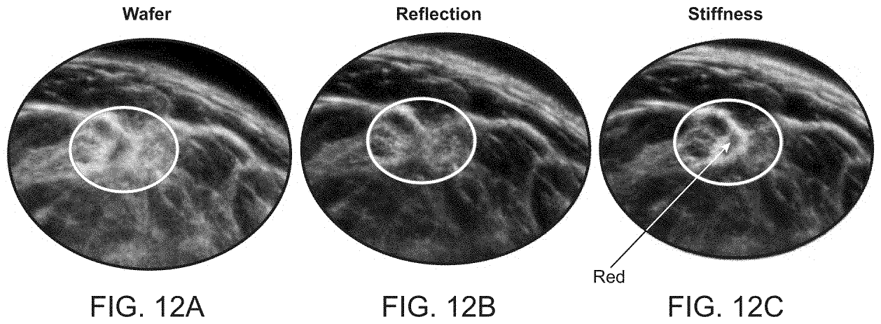

A morphological metric may comprise a size of a lesion. For example, a region may be larger or smaller than a threshold value. For example, a region may be larger or smaller than about 5 cm, about 2 cm, 1 cm, about 0.5 cm, about 0.1 cm or less. There is a likelihood of missing small cancers in imaging modalities such as mammography. Size of the cancerous tissue or the volume of tissue may be another parameter on a system for aiding a user to classify a volume of tissue. In some cases, a classification may depend on size. For example, various parameters may have differing associated probabilities based on whether a potential lesion is large or small. Large cancers may present a variation of parameters compared to small cancers. Some examples include: large cancers may appear black or gray or dark on wafer image or reflection image as shown in example of FIG. 11A and FIG. 11B; large cancers may persist between wafer image and reflection image; large cancers may appear blue or green on stiffness images due to necrosis, as shown in example of FIG. 11C; small cancers may disappear or blend in with the surrounding parenchyma on reflection images as shown in example of FIG. 12A and FIG. 12B; small cancers may not persist between wafer and reflection images; small cancers may be very stiff; small cancers may appear red or orange on stiffness images as shown in the example of FIG. 12C; etc.

A parameter of a set of parameters of an image or data set may comprise an average value of a sound propagation metric within a tumor, a kurtosis value within a tumor, a difference between a kurtosis values within a tumor and a kurtosis value within a peri-tumor, a standard deviation of a grayscale within a tumor, a gradient of a grayscale image within a tumor, a standard deviation of a gradient within a peri-tumor, a skewness of a gradient within a peri-tumor, a kurtosis of a corrected attenuation within a peri-tumor, a corrected attenuation of an energy within a tumor, a contrast of a grayscale of an image within a peri-tumor, a homogeneity of a grayscale of an image within a peri-tumor, or a difference in contrast of a grayscale within a tumor and within a peri-tumor.

A parameter of a set of parameters of an image or data set may comprise order statistics (i.e. mean, variance, skewness, kurtosis, contrast, noise level, signal to noise ratio (SNR), etc.) of the underlying acoustic parameters (the raw pixel value of each image) or the gray/color scale counter parts.

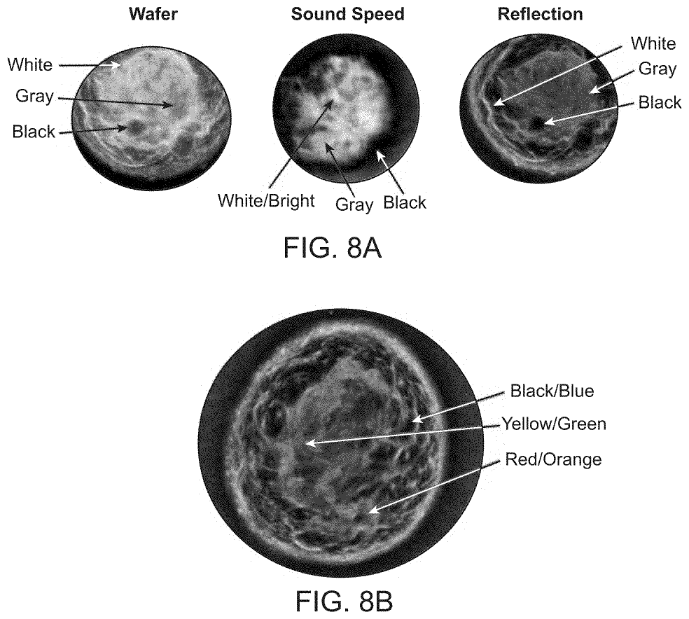

In some cases, the texture of the images can be assessed by using order statistics of histograms characterizing the value of grayscale distributions. Grayscale is a collection of the range of monochromatic (gray) shades. Grayscale may range from white to black. In terms of luminescence, grayscale may range from bright to dark. In some embodiments, features herein include texture features such as first order histogram features. In some embodiments, the features include higher order features which further characterize texture such as gray level co-occurrence matrices (GLCM) and their respective scalar features (energy, entropy, etc.) In some embodiments, the co-occurrence matrix herein is method which compares the intensity of a pixel with its local neighborhood. In some embodiments, the co-occurrence matrix can examine the number of times a particular value (in grey scale) co-occurs with another in some defined spatial relationship. Example of FIG. 8A shows how the range of grayscale shades may apply to wafer, sound speed and reflection images.

In some cases, the images may be assessed using parameters derived from colorscale images. Colorscale may relate to renderings of ultrasound images such as stiffness images that can be shown in color. These images may be representative of stiffness properties of volume of tissue such as breast tissue. The color map may range from black to red. In some embodiments, the color map may range from blue to red. Other color ranges may be defined for the volume of tissue representing a range of stiffness parameters. The stiffer the tissue, the closer the color may be to the red end of the color range. The color black or blue may be indicative of no stiffness or absence of stiffness. In some cases, these parameters may be derived from order statistics of histograms characterizing the value of grayscale distributions. Example of FIG. 8B shows how the range of colorscale shades may apply to stiffness images.

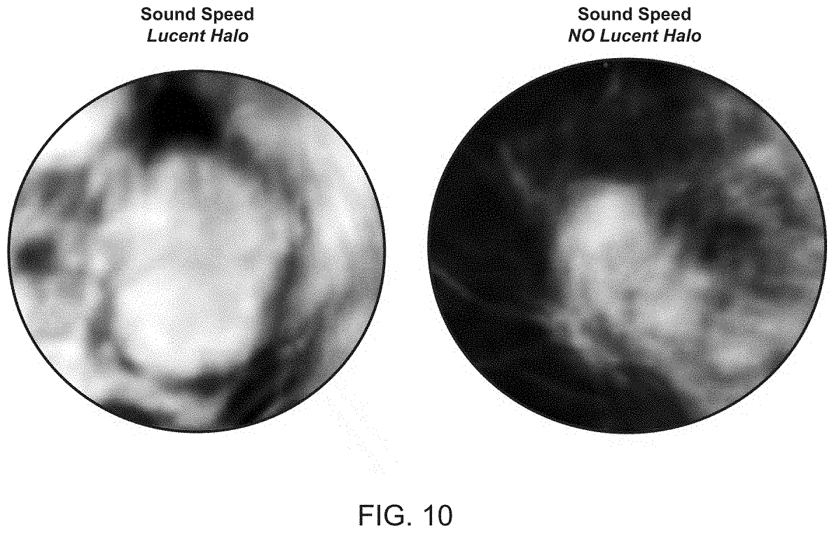

A parameter of a set of parameters in an image or data set may comprise a “lucent halo.” In some sound speed images, dark rings (lucent halos) may surround the volume of tissue such as breast tissue. Lucent halo may be observed in some fibroadenomas or cysts. Lucent halos may be indicative of benign process. In the example of FIG. 10 the fibroadenoma is encapsulated by lucent halo whereas the cancerous tissue is not.

A parameter of a set of parameters an image or data set may comprise a kurtosis value. A kurtosis value can describe or represent the sharpness of a peak of a frequency-distribution curve. In some cases, kurtosis can be calculated as a kurtosis of a gradient of an image such as a grayscale image. Kurtosis can be determined for any type of image, for example, a corrected attenuation image, an enhanced reflection image, a compounded enhanced reflection image, a sound speed image, etc.

A parameter of a set of parameters an image or data set may comprise a contrast. A contrast can be a measure of a difference in signal within a ROI such as a tumor or a peri-tumor.

A parameter of a set of parameters an image or data set may comprise a homogeneity value. A homogeneity value can be a measure of variation within a ROI such as a tumor or a peri-tumor.

A parameter of a set of parameters an image or data set may comprise at least one texture metric of a ROI. A texture metric can comprise at least one of an edgeness, a grey level co-occurrence matrix, and a Law's texture map.

A parameter of a set of parameters an image or data set may comprise a parameter of a wavelet of an image. In some cases, a wavelet of an image can represent an image. In some cases, a wavelet can be employed in the analysis of an image. Examples of wavelets can include a continuous wavelet transform of an image or a discrete wavelet transform of an image.

A parameter of a set of parameters an image or data set may comprise a standard deviation of an eroded grayscale image within a tumor, an average of an eroded grayscale image within a tumor, a standard deviation of an eroded grayscale image within a peri-tumor, a first order entropy of a gradient within a tumor, a first order mean of a gradient within a tumor, a difference between a first order entropy within a tumor and a first order entropy within a peri-tumor, a contrast within a tumor, a correlation within a tumor, a difference in contrast between a tumor and a peri-tumor, or a difference in homogeneity between a tumor and a peri-tumor.

A parameter of a set of parameters an image or data set may comprise one or more of the margin boundary score, the mean enhanced reflection, the relative mean of the enhanced reflection interior and exterior to the ROI, the standard deviation of the enhanced reflection, the mean sound speed, the relative mean sound speed interior and exterior to the ROI, the standard deviation of the sound speed, the mean attenuation, the standard deviation of the attenuation, the mean of the attenuation corrected for the margin boundary score, and the standard deviation of the attenuation corrected for the margin boundary score.

A parameter of a set of parameters an image or data set may comprise one or more of an irregularity of a margin, an average of sound speed values within a tumor, an average attenuation value within a peri-tumor, a contrast texture property of reflection within a peri-tumor, a difference between an average reflection value within a tumor and an average reflection value within a peri-tumor, a contrast texture property of a reflection within a tumor, a first order standard deviation of a sound speed value within a tumor, an average of a reflection value within a tumor, an average of a reflection value within a peri-tumor, a first order average of a reflection value within tumor, a difference between a homogeneity texture property of a reflection within a tumor and within a peri-tumor, a first order average of a sound speed value within a peri-tumor difference between a contrast texture property of an attenuation within a tumor and a contrast texture property of an attenuation within a peri-tumor, a standard deviation of a wavelet detail coefficient of a sound speed margin, a standard deviation of a wavelet detail coefficient of a reflection margin, a histogram of entropy of a wavelet detail coefficient of a reflection margin, a local minimum standard deviation of a wavelet detail coefficient of reflection margin, or a maximum of a standard deviation of a crisp contrast.

As described herein above, each image may comprise a portion of a rendering. For example, a rendering may be formed from one or more “stacks” of 2D images corresponding to a series of “slices” of the volume of tissue for each measured acoustomechanical parameter at each step in a scan of the volume of tissue. Each slice may comprise an image or layer of the rendering. Each layer, subset of layers, classification of layers, and/or ROI may have one or many associated parameters, for example, any type of parameter associated with image characteristics as described herein.

A parameter of a set of parameters an image or data set may comprise volumetric parameters. A volumetric parameter may be derived from a plurality of image positions along an anterior-exterior axis of tissue. In some cases, a volumetric parameter may be a qualitative volumetric parameter. In some cases, a volumetric parameter may be a quantitative volumetric parameter.

In an example of a qualitative volumetric parameter, a user may indicate whether or not a region of interest “flows” from layer to layer of the stack of 3D images. The parameter “flows” may be used to distinguish dense tissue from a lesion, or mass. The concept of flowing parenchyma may relate to mass detection. Dense breast tissue may flow like passing clouds in a series of images whereas, as mass may appear in one or a subset of images. If an area of the volume of tissue flows or changes shape from slice to slice the likelihood of that volume being a mass may be small.

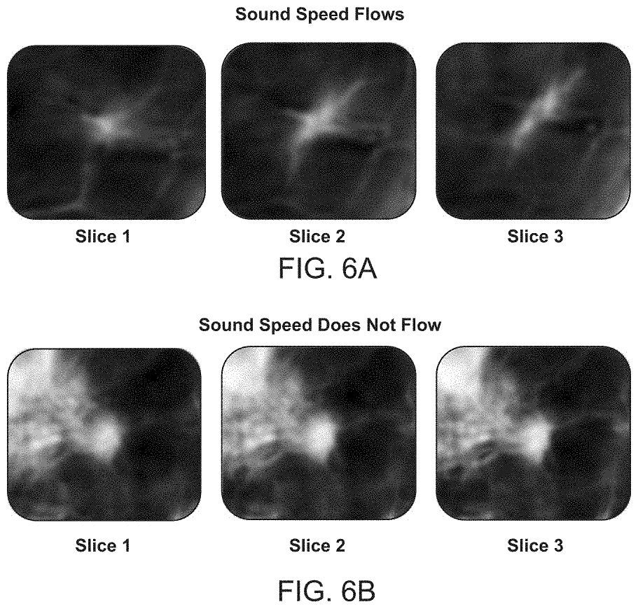

FIG. 6A shows an example of tissue flowing in a series of sound speed images. FIG. 6B shows an example of a non-flowing mass in a series of sound speed images. As shown, dense breast tissue flows (e.g., irregularly changes shape or even disappears) from slice to slice as one scrolls through the breast while viewing a stack of images, whereas a lesion, or mass, will remain in an image or uniformly change shape through multiple image slices.

A “flows” parameter may be integrated into a method for classifying a lesion within a volume of tissue. For example, a method of classifying a lesion within a volume of tissue may comprise receiving from an ultrasound transducer a first speed rendering at a first anterior-posterior position, the first speed rendering comprising sound speed data within the volume of tissue. A method of classifying a lesion within a volume of tissue may further comprise identifying a region of interest within the first speed rendering. A method of classifying a lesion within a volume of tissue may further comprise receiving from the ultrasound transducer a second speed rendering at a second anterior-posterior position, the second speed rendering comprising sound speed data within the volume of tissue. A method of classifying a lesion within a volume of tissue may further comprise identifying a second region of interest within the second speed rendering. A method of classifying a lesion within a volume of tissue may further comprise classifying a lesion within the volume of tissue based on a similarity or lack thereof of the first region of interest and the second region of interest.

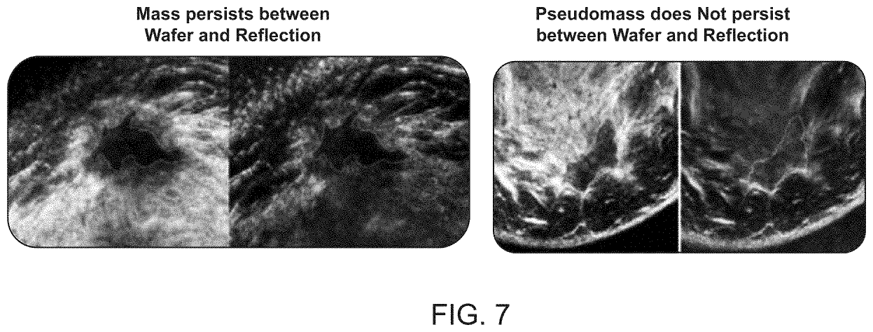

In an example of a qualitative volumetric parameter, a user may indicate whether or not a region of interest “persists” from image type to image type in a plurality of image types. For example, a persist parameter may relate to a mass appearing in more than one ultrasonic image rendering, for example, appearing in both reflection and wafer images. If a mass only appears in one image type, for example, only in a reflection image, the likelihood of that volume of tissue being a real mass may be small. Dark areas in reflection images may generally represent normal tissue. In some cases, if a mass is small in size for example smaller than 1 cm, the mass may not persist between different image types. As shown in the example of FIG. 7, the normal volume of tissue (pseudomass) may not persist between two image sequence types.

A “persists” parameter may be integrated into a method for classifying a lesion within a volume of tissue. For example, a method of classifying a lesion within a volume of tissue may comprise receiving from an ultrasound transducer at least one reflection rendering comprising sound reflection data within the volume of tissue. A method of classifying a lesion within a volume of tissue may further comprise identifying a region of interest within the at least one reflection rendering. A method of classifying a lesion within a volume of tissue may further comprise receiving from the ultrasound transducer at least one combined rendering comprising sound speed data and sound reflection data within the volume of tissue. A method of classifying a lesion within a volume of tissue may further comprise identifying a second region of interest within the at least one combined rendering. A method of classifying a lesion within a volume of tissue may further comprise classifying a lesion within the volume of tissue based on a similarity or lack thereof of the first region of interest and the second region of interest.

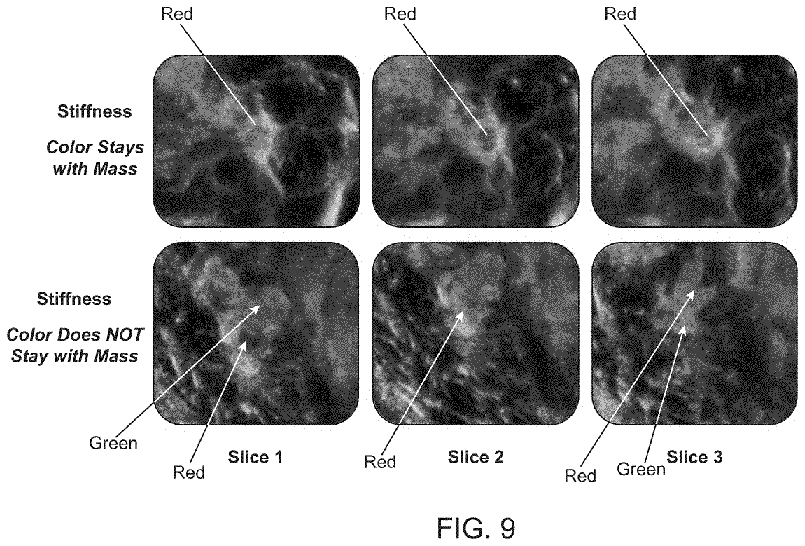

In an example of a qualitative volumetric parameter, a user may indicate whether or not a region of interest “stays” on a colorscale image. The parameter “stays” may be used to distinguish dense tissue from a lesion, or mass. In general, it relates to the color staying as the user scrolls through stiffness images in a stiffness image sequence, for example, a sequence that varies by image depth among a series of layers. The color associated with dense tissue may not stay and may pass like a cloud flow as the user scroll through a series of stiffness images. The likelihood of the color staying is high with the cancerous masses. The top section of FIG. 9 shows how color may stay with mass in a sequence of stiffness images. The bottom section of FIG. 9 shows how color passes in a sequence of stiffness images for a dense volume of tissue image.

A “stays” parameter may be integrated into a method for classifying a lesion within a volume of tissue. For example, a method of classifying a lesion within a volume of tissue may comprise receiving from an ultrasound transducer a first stiffness rendering at a first anterior-posterior position, the first stiffness rendering comprising a combination of sound speed and sound attenuation data within the volume of tissue. A method of classifying a lesion within a volume of tissue may further comprise identifying a region of interest within the first attenuation rendering. A method of classifying a lesion within a volume of tissue may further comprise receiving from the ultrasound transducer a second attenuation rendering at a second anterior-posterior position, the second attenuation rendering comprising a second combination of sound speed and sound attenuation within the volume of tissue. A method of classifying a lesion within a volume of tissue may further comprise identifying a second region of interest within the second attenuation rendering. A method of classifying a lesion within a volume of tissue may further comprise classifying a lesion within the volume of tissue based on a similarity or lack thereof of the first region of interest and the second region of interest.

A set of parameters can include at least 1, 2, 3, 4, 5, 6, 7, 8, 9, 10, 11, 12, 13, 14, 15, 16, 17, 18, 19, 20, 50, 100, 1000, or more parameters.

Other parameters, methods of extraction, and methods of use thereof are described in International Patent Application PCT/US2019/029592, which is incorporated herein by reference in its entirety.

### Potential Classifications

Systems for aiding a user to classify a volume of tissue disclosed herein, may comprise a plurality of probabilities each associated with a potential classification of a region of the volume of tissue. The classifications may comprise particular aspects of the type of tissue, such as to determine whether a mass in the tissue may be a tumor, cyst, fibroadenoma, or other kind of mass. Systems for aiding a user to classify a volume of tissue disclosed herein may be used to characterize the tissue to facilitate diagnoses of cancer, assess its type, and determine its extent (e.g., to determine whether a mass in the tissue may be surgically removable), or to assess risk of cancer development (e.g., measuring breast tissue density). Potential classifications include but are not limited to soft dense tissue, stiff dense tissue, fatty lobules, cysts, small cysts, soft fibroadenomas, stiff fibroadenomas, cancers, non-specific benign masses, and unidentifiable tissue. Various classifications and illustrative images thereof are included herein.

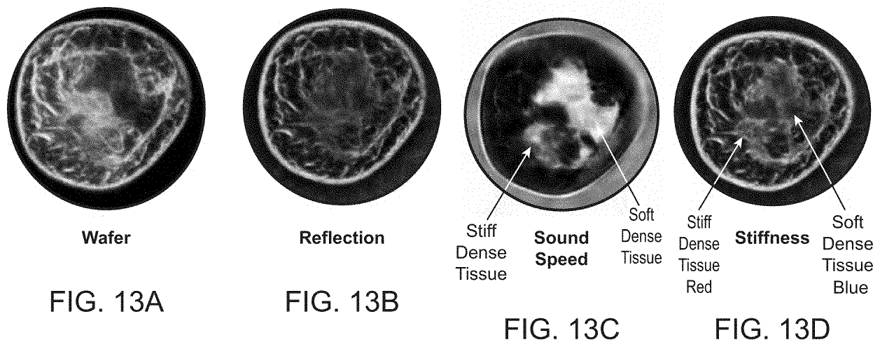

Soft dense tissue may flow as the user scrolls in a sequence of images. Soft dense tissue may appear dark, black or gray on wafer images, such as shown in example of FIG. 13A. Soft dense tissue may appear white or bright on sound speed images, such as shown in example of FIG. 13C. Soft dense tissue may appear dark or gray on reflection images, such as shown in example of FIG. 13B. Since there may be an absence of stiffness, soft dense tissue may appear black or blue on stiffness images, such as shown in example of FIG. 13D.

Stiff dense tissue may flow as the user scrolls in a sequence of images. Stiff dense tissue may appear white or gray on wafer images, such as shown in example of FIG. 13A. Stiff dense tissue may appear white or bright on sound speed images, such as shown in example of FIG. 13C. Stiff dense tissue may appear white on reflection images, such as shown in example of FIG. 13B. Stiff dense tissue may appear green or yellow or orange or red on stiffness images, such as shown in example of FIG. 13D.

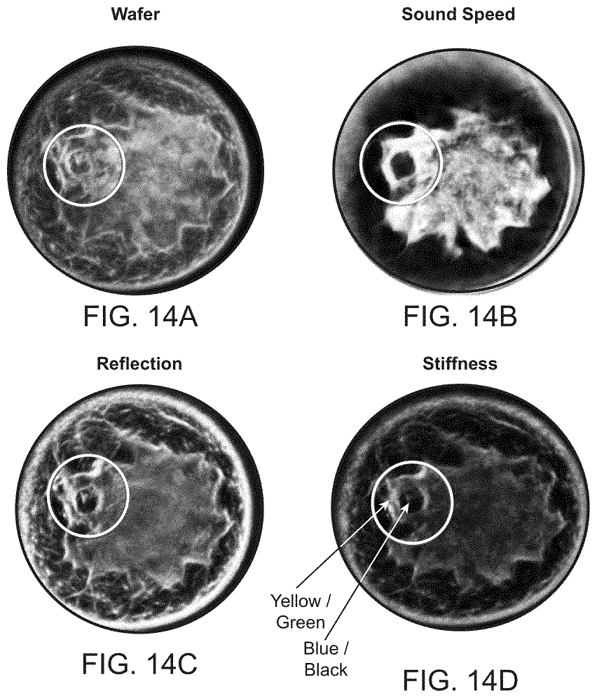

Fatty tissue may appear darker than the surrounding tissue on wafer and reflection images. Fatty tissue may also appear dark or black on sound speed images because of its low density. Fat may have low stiffness on stiffness images. Fatty tissue may appear as round or oval shaped with circumscribed margins. A fatty lobule may appear white or gray on wafer images, such as shown in example of FIG. 14A. A fatty lobule may appear dark or black on sound speed images, such as shown in example of FIG. 14B. A fatty lobule may appear black on reflection images, such as shown in example of FIG. 14C. A fatty lobule may appear black or blue on stiffness images, since it is soft and may not have dense properties, such as shown in example of FIG. 14D.

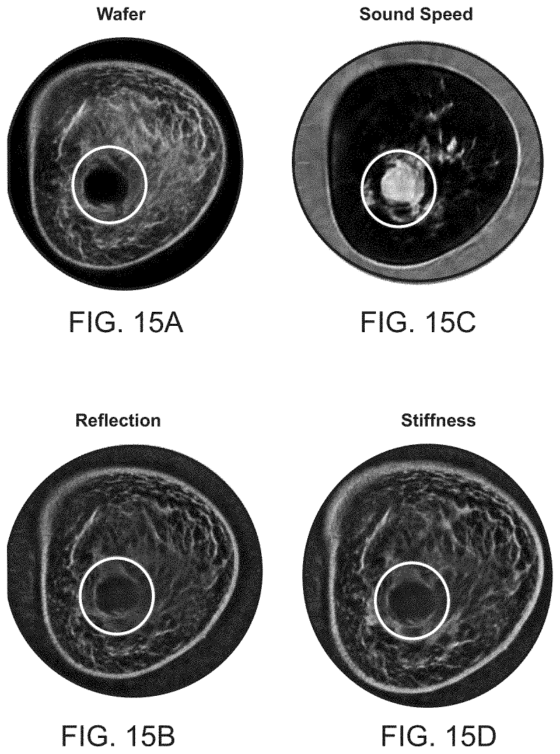

A cyst mass may appear as round or oval shaped with circumscribed margins. A cyst may appear black or gray on wafer and/or reflection images, such as shown in examples of FIG. 15A and FIG. 15C respectively. A cyst may appear gray or white or bright on sound speed images similar to the surrounding water, such as shown in example of FIG. 15B. In some cases, a cyst mass may have a lucent halo. A cyst may appear black or blue on stiffness images, since it is soft and may lack stiffness properties, such as shown in example of FIG. 15D. A cyst mass may be surrounded by dense parenchyma.

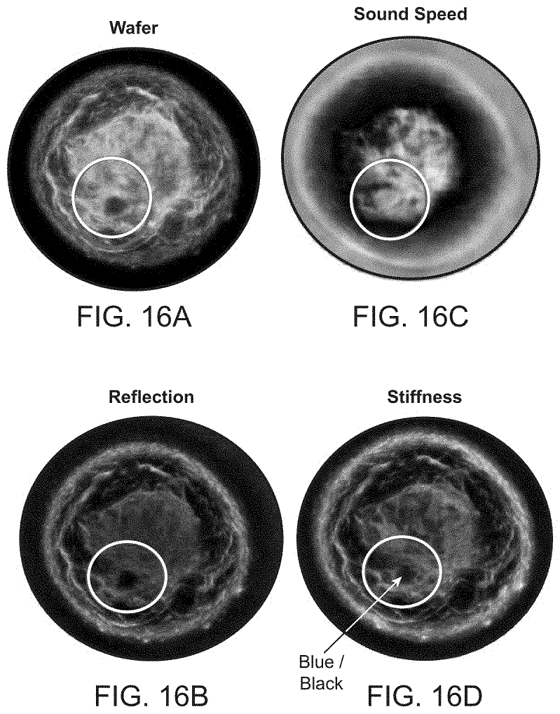

If the size of the cyst is smaller than 1 cm (hereinafter referred to as small cyst), the assessment of the cyst may be different from another cyst. A small cyst mass may appear as round or oval shaped with indistinct margins. A cyst may appear black or gray on wafer and/or reflection images, such as shown in examples of FIG. 16A and FIG. 16C respectively. A small cyst may appear gray or white or bright on sound speed images similar to the surrounding water, such as shown in example of FIG. 16B. In some cases, a small cyst mass may have a lucent halo. A small cyst may appear stiff or yellow or green on stiffness images, such as shown in example of FIG. 16D.

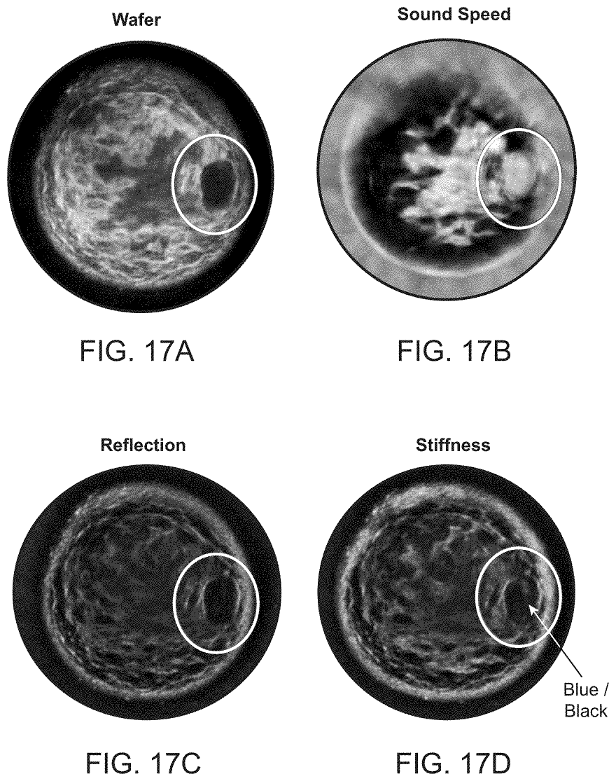

Soft fibroadenomas may appear circumscribed round or oval shape. Soft fibroadenomas may appear as black on wafer images, such as shown in example of FIG. 17A. Soft fibroadenomas may appear as white or bright on sound speed images, such as shown in example of FIG. 17B. Soft fibroadenomas may have a lucent halo surrounding the mass. Soft fibroadenoma may appear as black or gray on reflection images, such as shown in example of FIG. 17C. Soft fibroadenoma may exhibit a range of color from blue to red depending on the stiffness properties on the stiffness images. In the example of FIG. 17D the soft fibroadenoma is shown in blue with a bit of green around the periphery.

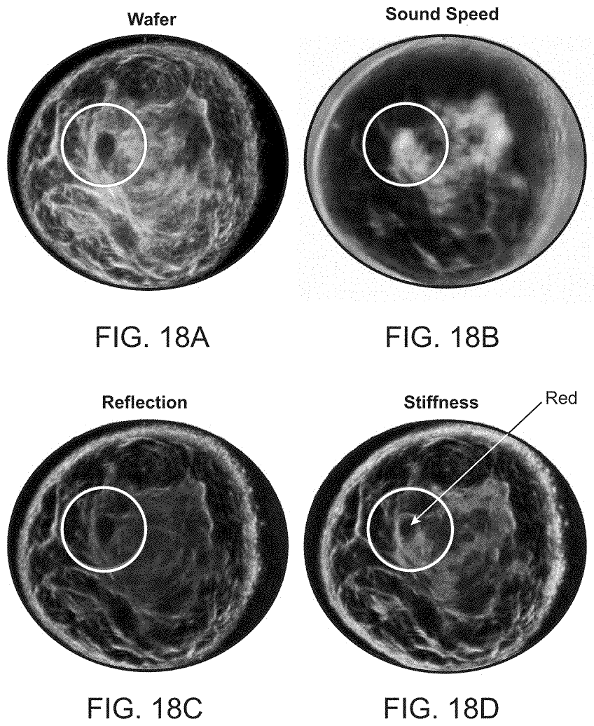

Smaller or stiff fibroadenomas may exhibit features mimicking cancer. Stiff fibroadenomas may appear as a round or oval mass with indistinct margins. Stiff fibroadenomas may appear as black on wafer images, such as shown in example of FIG. 18A. Stiff fibroadenomas may appear as white or bright on sound speed images, such as shown in example of FIG. 18B. Stiff fibroadenomas may appear as black or gray on reflection images, such as shown in example of FIG. 18C. Stiff fibroadenomas may appear as green, yellow, orange or red on the stiffness images, such as shown in example of FIG. 18D.

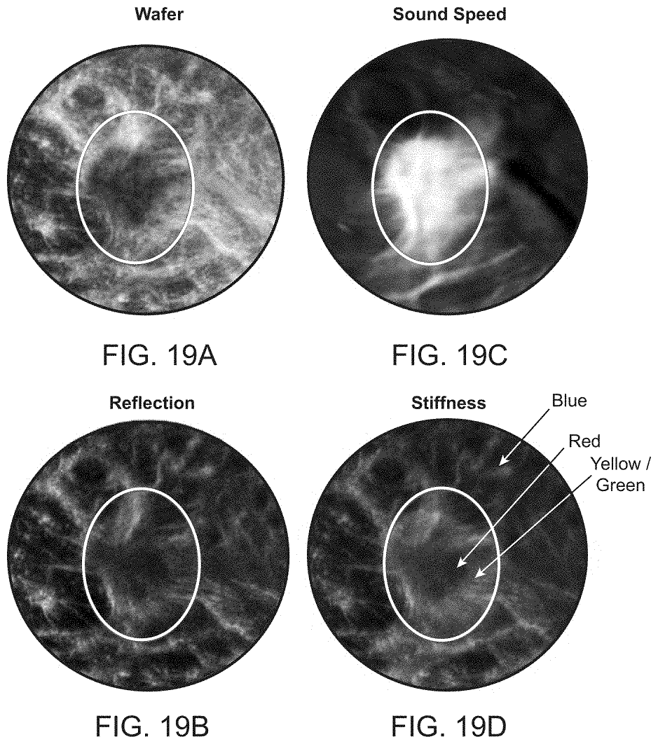

Cancers may exhibit a range of characteristics. In general, cancers may have indistinct or spiculated margins. Cancers may be irregular in shape. They may also be round or oval in shape. Cancers may appear black or gray in wafer images, such as shown in example of FIG. 19A. Cancers may appear black or gray on reflection images depending on the size of the cancer, such as shown in example of FIG. 19B. Cancers may appear white or bright on sound speed images, such as shown in example of FIG. 19C. Cancer appearance may vary on stiffness images depending on the stiffness of the mass or the size of the mass. An example of cancer on stiffness image is provided in FIG. 19D.

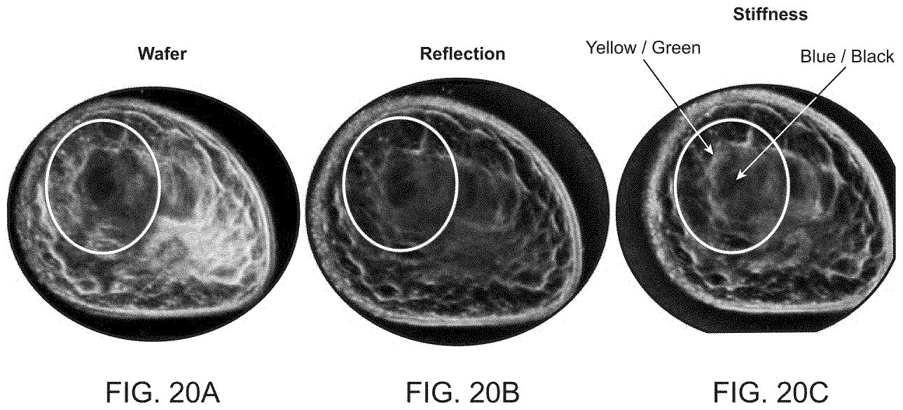

Large cancers may appear black or gray on wafer and reflection images, such as shown in examples of FIG. 20A and FIG. 20B respectively. Large cancers may persist between wafer images and reflection images. Large cancers may appear soft blue or green on stiffness images, such as shown in example of FIG. 20C. The color may be due to necrosis. Even when soft, cancers may be stiff along the peritumoral regions, and may be heterogeneously soft internally.

Small cancers may disappear or blend in with surrounding parenchyma on reflection images, such as shown in example of FIG. 12B. Small cancers may not persist between wafer and reflection images. Small cancers may be very stiff or orange or red on the stiffness images, such as shown in example of FIG. 12C.

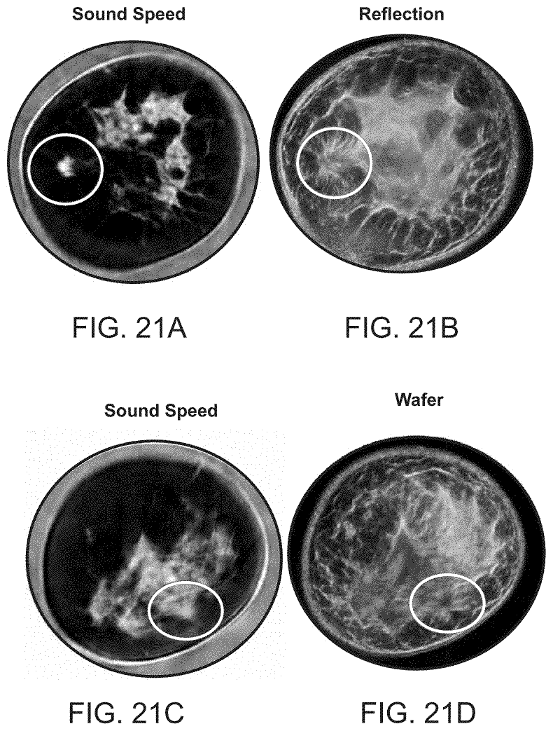

Cancers may be easier to detect in sound speed images compared to reflection images. Spiculations may be easier to detect in sound speed images compared to reflection images. In sound speed images, cancers may be white or bright relative to the surrounding parenchyma, such as shown in example of FIG. 21A. Cancers may be usually irregular in shape and may present spiculations. On reflection images, cancers may present as dark or be similar to surrounding tissue, such as shown in example of FIG. 21B.

Sound speed may have higher sensitivity and may be used within wafer images to suppress fat. Sound speed images may have the best view of mass margins and spiculations. Sound speed margin evaluation may be more useful than wafer alone. Examples of margins in sound speed image and wafer image are shown FIG. 21C and FIG. 21D respectively.

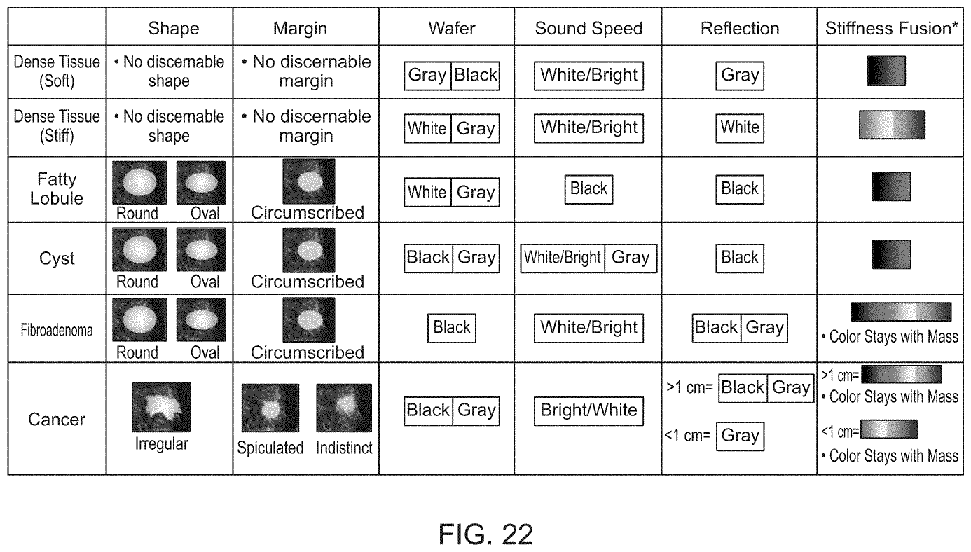

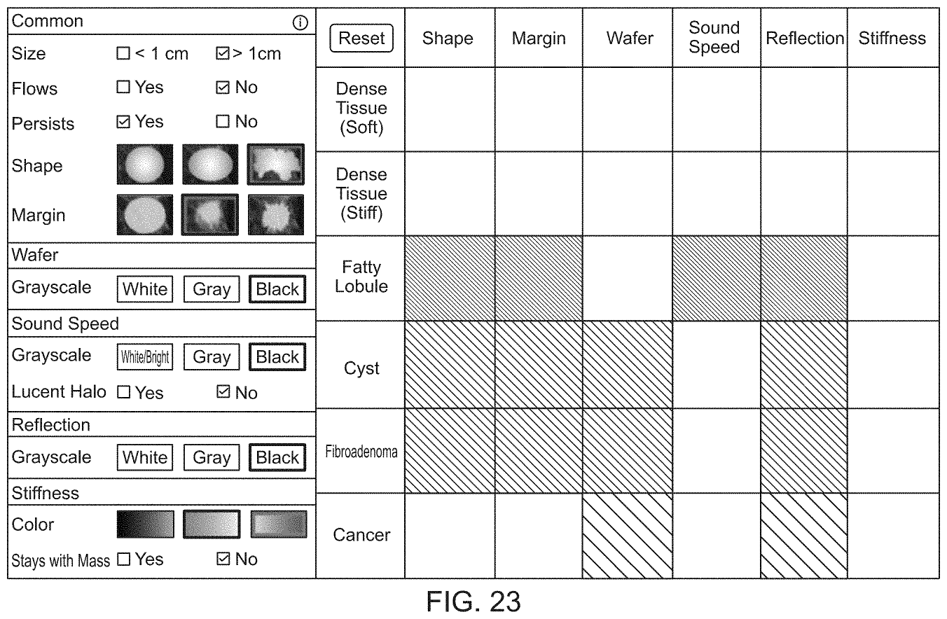

FIG. 22 summarizes lesion characteristics and the parameters related to them in a matrix form table.

### Probability Engine

The systems and methods for aiding a user to classify a volume of tissue disclosed herein, may comprise a plurality of probabilities each associated with a potential classification of a region of the volume of tissue. The classifications may be among the characteristics described above. The image classification may be related to one or more parameters of the plurality of parameters, described above. The plurality of parameters may be assumed to be independent of one another.

As shown in FIG. 2, a graphical representation of a system for aiding a user to classify a volume of tissue may comprise a matrix style display. The rows or columns of the matrix may comprise all or a subset of the plurality of potential classifications of the image. The columns or rows may comprise the subset of relevant parameters of the plurality of parameters. An element of the matrix, hereinafter referred to as “cell”, may provide a visible representation of a probability of a potential classification associated with the parameter of the subset of relevant parameters. The parameter selection panel (CSP) and/or characteristics matching panel (CMP) may be visible to the user on a user interface of an electronic device such as but not limited to a tablet or a smart phone.

A probability engine may comprise the mathematical logic that computes the probabilities for each element of the graphical display, for example, each cell of the CMP. These probabilities may be computed based on at least two input types described below.

In a first input type, a probability of the plurality of probabilities may be a probability that one or more parameters are observed for given a tissue and/or mass type. This conditional probability may be denoted as P(G|T), where G denotes the group of parameters, and T denotes the tissue and/or mass type.

For example, the probability that a set of sound speed image parameters may include (flows=yes, persists=yes, sound speed grayscale=black, lucent halo=yes) given that the observed mass type is cancer, is denoted as P(G_{SS}=(yes, yes, black, yes)|T=cancer).

Table 1 lists several examples of considered subsets of parameters, the parameters they include, and the notation used. In some cases, the characteristic ‘flows’ and ‘persists’ may be common to all groups, as described herein. Each group may correspond to a column of the CMP as shown in FIG. 2. The conditional probabilities may be estimated based on observations for a large database of images.

In some cases, all characteristics may be assumed to be independent of one another, conditionally on the tissue and/or mass type. In some cases, all characteristics except flows and persists can be assumed to be independent of each other, conditionally on the tissue and/or mass type. In some cases, the pair (flows, persists) may described using a joint distribution. The conditional probability of a group may be expressed as the product of the probabilities of its independent components.

TABLE 1
Parameter Groups

| Group | Included Characteristics | Notation |
|---|---|---|
| Shape | Flows, Persists, Shape | Gₛ |
| Margin | Flows, Persists, Margin | Gₘ |
| Wafer | Flows, Persists, Wafer Grayscale | G_{w} |
| Sound | Flows, Persists, Sound Speed Grayscale, Lucent | Gₛₛ |
| Speed | Halo | |
| Reflection | Flows, Persists, Reflection Grayscale | Gᵣ |
| Stiffness | Flows, Persists, Stiffness Colorscale, Stays with | G_{sf} |
| | mass | |

The cell probability may be computed as the probability that a specific tissue and/or mass type is observed for a subset of the plurality of parameters. In terms of conditional probability, this may be denoted as P(T|G). In other words, the cell probability associated with the potential classification may be a reverse conditional probability.

For example, the probability that the observed mass type is cancer given that the sound speed characteristics are (flows=yes, persists=yes, sound speed grayscale=black, lucent halo=yes), is given by P(T=cancer|G=(yes, yes, black, yes)).

The cell probability (reverse conditional probability) may be computed using Bayes' formula as:

$$P ( T = t \vert G = g ) = \frac{P ( G = g \vert T = t ) P ( T = t )}{P ( G = g )} = \frac{P ( G = g \vert T = t ) P ( T = t )}{{Σ}_{{t}^{′}} P ( G = g \vert T = {t}^{′} ) P ( T = {t}^{′} )}$$

where the sum in the denominator is over all possible tissue and/or mass types. In some cases, the types may be assumed to be uniformly distributed, i.e., P(T=t)=⅙ for all 6 types (soft dense tissue, stiff dense tissue, fatty lobule, cyst, fibroadenoma, cancer).

The sum of P(T=t|G=g) over all types is equal to one (1). Using Bayes' formula,

$$\begin{matrix}{\sum }_{t} P ( T = t \vert G = g ) = {\sum }_{t} \frac{P ( G = g \vert T = t ) P ( T = t )}{P ( G = g )} \\ = \frac{1}{P ( G = g )} {\sum }_{t} P ( G = g \vert T = t ) P ( T = t ) \\ = \frac{P ( G = g )}{P ( G = g )} \\ = 1\end{matrix}$$

In other words, for a given column, the cell probabilities over all rows may add up to one (1).

The row probability may be computed as the average of the cell probabilities for the cells of that row. As such, the row probabilities may also add up to one (1), i.e., they may describe a row probability distribution.

The probability of potential classification may be visualized in different ways. In the example of FIG. 2 the probabilities of each cell are shown by different saturation of a color such as blue color. The stronger intensity of the color may show higher probability and the weaker intensity of the color may show smaller probability of a classification. Other examples of visualization of probabilities on a graphical display may be, score value or size variation of a visual marker such as a bar or a gray scale color variation. In the example of FIG. 2 the rows are representative of the lesion types (characteristics) and the columns are representative of different ultrasonic renderings or their combination. At least one image may be selected from the group consisting of an enhanced reflection image, a B-mode reflection image, a sound speed image, and a stiffness representation.

Classification of the lesion (tissue) may comprise a cancer, a fibroadenoma, a cyst, a nonspecific mass or an unidentifiable mass. The rows or the columns of the matrix in the graphical display may represent the lesion type.

The framework described above may be supplemented with characterization rules. These rules may modify the row probability distribution computed above based on overriding characterization principles, hence modifying the color of the highlighted cells.

For example, a black sound speed grayscale may be a strong indicator of a fatty lobule, even if other individual cell probabilities are low. Conversely, if the sound speed grayscale is not black, a fatty lobule is highly unlikely, even if other individual cell probabilities are high. As shown in the example of FIG. 23, for sound speed image grayscale parameter black is selected by the user. As described, this may be a strong indication of a fatty lobule. The row indicating fatty lobule may therefore be darkened to indicate high likelihood. The systems and methods for aiding a user to classify a volume of tissue may implement a mechanism that increases/decreases by a fixed factor, the row probabilities associated with matching characterization rules, hence darkening/lightening the color associated with these rows.

As described herein, obtaining the probabilities of the systems and methods of the present disclosure, i.e. the probability of a certain classification (e.g. cancer) given a set of parameters, may utilize the knowledge of reverse conditional probability. In other words, a stored dataset of the likelihood of occurrence of a set of parameters, given a certain classification (e.g. cancer) may be beneficial. The stored dataset may be stored in the form of a library of features in a local storage or on a server such as a cloud server.

### Digital Processing Device

In some embodiments, the platforms, systems, media, and methods described herein include a digital processing device, or equivalent, a processor. In further embodiments, the processor includes one or more hardware central processing units (CPUs) or general purpose graphics processing units (GPGPUs) or tensor processing unit (TPU) that carry out the device's functions. In still further embodiments, the digital processing device further comprises an operating system configured to perform executable instructions. In some embodiments, the digital processing device is optionally connected to a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the processor includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

In some embodiments, the processor includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the processor is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing-based storage. In further embodiments, the storage and/or memory device is a combination of devices such as those disclosed herein.

In some embodiments, the processor includes a display to send visual information to a user. In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In yet other embodiments, the display is a head-mounted display in communication with the processor, such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In some embodiments, the processor includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

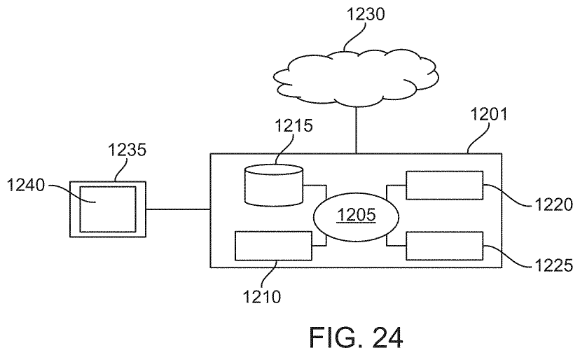

Referring to FIG. 24, in a particular embodiment, an example processor 1201 is programmed or otherwise configured to allow presentation of several images of volume of tissue, selection of parameters, storage of parameters, calculation of probabilities, classification of regions of volume of tissue, etc. The processor 1201 can regulate various aspects of the present disclosure, such as, for example, probability calculation, image classification, etc. In this embodiment, the processor 1201 includes a central processing unit (CPU, also “processor” and “computer processor” herein) 1205, which can be a single core or multi core processor, or a plurality of processors for parallel processing. The processor 1201 also includes memory or memory location 1210 (e.g., random-access memory, read-only memory, flash memory), electronic storage unit 1215 (e.g., hard disk), communication interface 1220 (e.g., network adapter, network interface) for communicating with one or more other systems, and peripheral devices, such as cache, other memory, data storage and/or electronic display adapters. The peripheral devices can include storage device(s) or storage medium 1265 which communicate with the rest of the device via a storage interface 1270. The memory 1210, storage unit 1215, interface 1220 and peripheral devices are in communication with the CPU 1205 through a communication bus 1225, such as a motherboard. The storage unit 1215 can be a data storage unit (or data repository) for storing data. The processor 1201 can be operatively coupled to a computer network (“network”) 1230 with the aid of the communication interface 1220. The network 1230 can be the Internet, an internet and/or extranet, or an intranet and/or extranet that is in communication with the Internet. The network 1230 in some cases is a telecommunication and/or data network. The network 1230 can include one or more computer servers, which can enable distributed computing, such as cloud computing. The network 1230, in some cases with the aid of the device 1201, can implement a peer-to-peer network, which may enable devices coupled to the device 1201 to behave as a client or a server.

Continuing to refer to FIG. 24, the processor 1201 includes input device(s) to receive information from a user, the input device(s) in communication with other elements of the device via an input interface 1250. The processor 1201 can include output device(s) 1255 that communicates to other elements of the device via an output interface 1260.

Continuing to refer to FIG. 24, the memory 1210 may include various components (e.g., machine readable media) including, but not limited to, a random access memory component (e.g., RAM) (e.g., a static RAM “SRAM”, a dynamic RAM “DRAM, etc.), or a read-only component (e.g., ROM). The memory can also include a basic input/output system (BIOS), including basic routines that help to transfer information between elements within the processor, such as during device start-up, may be stored in the memory 1210.

Continuing to refer to FIG. 24, the CPU 1205 can execute a sequence of machine-readable instructions, which can be embodied in a program or software. The instructions may be stored in a memory location, such as the memory 1210. The instructions can be directed to the CPU 1205, which can subsequently program or otherwise configure the CPU 1205 to implement methods of the present disclosure. Examples of operations performed by the CPU 1205 can include fetch, decode, execute, and write back. The CPU 1205 can be part of a circuit, such as an integrated circuit. One or more other components of the device 1201 can be included in the circuit. In some cases, the circuit is an application specific integrated circuit (ASIC) or a field programmable gate array (FPGA).

Continuing to refer to FIG. 24, the storage unit 1215 can store files, such as drivers, libraries, and saved programs. The storage unit 1215 can store user data, e.g., user preferences and user programs. The processor 1201 in some cases can include one or more additional data storage units that are external, such as located on a remote server that is in communication through an intranet or the Internet. The storage unit 1215 can also be used to store operating system, application programs, and the like. Optionally, storage unit 1215 may be removably interfaced with the processor (e.g., via an external port connector (not shown)) and/or via a storage unit interface. Software may reside, completely or partially, within a computer-readable storage medium within or outside of the storage unit 1215. In another example, software may reside, completely or partially, within processor(s) 1205.

Continuing to refer to FIG. 24, the processor 1201 can communicate with one or more remote computer systems 1202 through the network 1230. For instance, the device 1201 can communicate with a remote computer system of a user. Examples of remote computer systems include personal computers (e.g., portable PC), slate or tablet PCs (e.g., Apple® iPad, Samsung® Galaxy Tab), telephones, Smart phones (e.g., Apple® iPhone, Android-enabled device, Blackberry®), or personal digital assistants.

Continuing to refer to FIG. 24, information and data can be displayed to a user through a display 1235. The display is connected to the bus 1225 via an interface 1240, and transport of data between the display other elements of the device 1201 can be controlled via the interface 1240.

Methods as described herein can be implemented by way of machine (e.g., computer processor) executable code stored on an electronic storage location of the processor 1201, such as, for example, on the memory 1210 or electronic storage unit 1215. The machine executable or machine readable code can be provided in the form of software. During use, the code can be executed by the processor 1205. In some cases, the code can be retrieved from the storage unit 1215 and stored on the memory 1210 for ready access by the processor 1205. In some situations, the electronic storage unit 1215 can be precluded, and machine-executable instructions are stored on memory 1210.

### Non-Transitory Computer Readable Storage Medium

In some embodiments, the platforms, systems, media, and methods disclosed herein include one or more non-transitory computer readable storage media encoded with a program including instructions executable by the operating system of an optionally networked processor. In further embodiments, a computer readable storage medium is a tangible component of a processor. In still further embodiments, a computer readable storage medium is optionally removable from a processor. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable in the processor's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

An example of the electronic device used for the systems methods disclosed herein is a tablet. The iLCM application may be executed automatically in kiosk mode when the tablet is powered up. This functionality may be implemented by locking the iLCM application at installation using a software tool such as SureLock.

In some embodiments, the CSP and CMP panels can be swapped by the user using a drag and drop operation or click operation or using a drop down menu for positioning the panels. The iLCM application may store the last configuration in the application preferences. In some cases, the iLCM Software may not read or write any other configuration parameters. In some cases, the iLCM Software can read or write other configuration parameters.

In some embodiments, the iLCM may not log any information. In some cases, the iLCM Software may log information. In some cases, the iLCM Software may not implement any user account. In some cases, the iLCM Software may implement one or a plurality of user accounts for a single or a subset of users. In some cases, the iLCM Software may not store any data on permanent storage. In some cases, the iLCM Software may store data on permanent storage.

An example of operating system may be Android. In some cases, the code may be developed in Java using the native Android API. The native Android API may be used in addition to cross-platform open source frameworks such as React Native (based on JavaScript) as it may be a safer option for long-term maintenance, uses strict language, which is less error prone, and provides full API access.

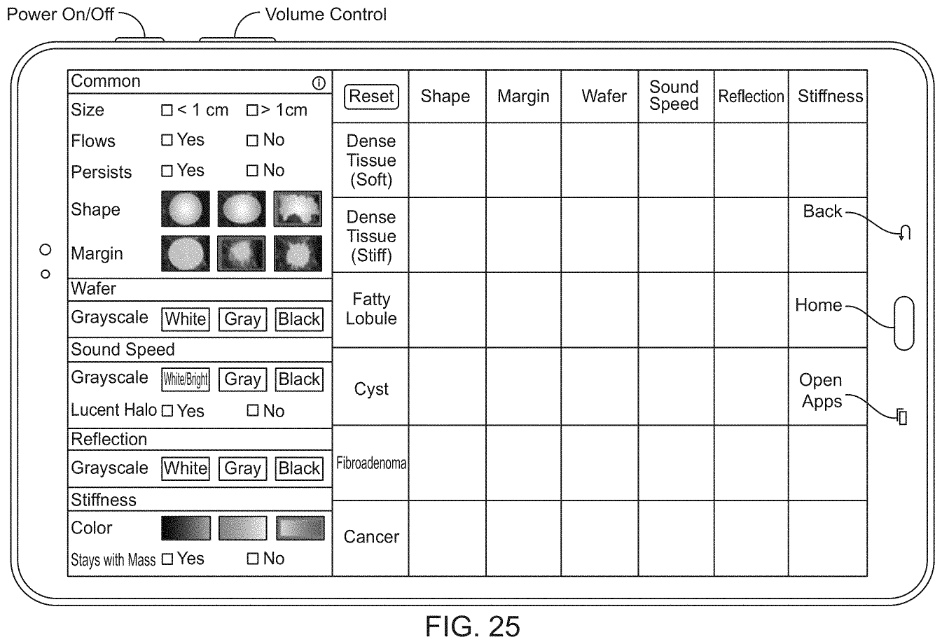

FIG. 25 shows an example of a tablet's physical controls used for implementing the methods and systems of the present disclosure. The power button may be enabled or specialized for the functions of the methods and systems described herein. Power On/Off button may allow the user to turn the screen off and on with a quick press. A long press may prompt the user to restart/power off the tablet. When the tablet is powered off, a long press may turn the tablet on and may proceed through the boot process.

In some embodiments, the user may be able to change selection of parameters. In some cases, a message may appear in the form of for example a pop-up window if the user makes a wrong selection, informing the user of the wrong selection of parameter or prompting user to make another selection. In some embodiments, there may be a reset button or mechanism for user to reset all or a subset of selections. The Lesion Characteristic Matrix's columns may list the categories of the input section, and the rows may contain lesion/tissue types. The display table may populate each of the grid squares, with a variable color, based on the likelihood of occurrence of the selected characteristics with a given lesion type.

In some embodiments, the user may be able to scroll within a plurality of images. In some cases, the user may be able to toggle between image types to compare the images. In some embodiments different image types for example renderings of ultrasound images such as sound speed and reflection may be shown next to each other or in other order for user to make comparisons. In some cases, the user may be able to zoom in or zoom out in the images to focus on certain features. In some embodiments, different image types may be presented in a certain order. In other cases, there may be no order in presentation of different images. In some embodiments, the user may select parameters from two or more images.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™ and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous JavaScript and XML (AJAX), Flash® ActionScript, JavaScript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

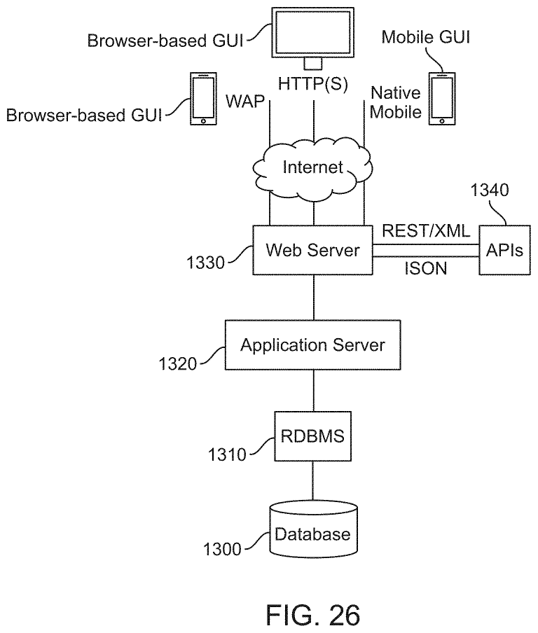

Referring to FIG. 26, in a particular embodiment, an application provision system comprises one or more databases 1300 accessed by a relational database management system (RDBMS) 1310. Suitable RDBMSs include Firebird, MySQL, PostgreSQL, SQLite, Oracle Database, Microsoft SQL Server, IBM DB2, IBM Informix, SAP Sybase, SAP Sybase, Teradata, and the like. In this embodiment, the application provision system further comprises one or more application severs 1320 (such as Java servers, .NET servers, PHP servers, and the like) and one or more web servers 1330 (such as Apache, IIS, GWS and the like). The web server(s) optionally expose one or more web services via app application programming interfaces (APIs) 1340. Via a network, such as the Internet, the system provides browser-based and/or mobile native user interfaces.

### Mobile Application

FIG. 25 shows an example of a mobile application used for implementing the methods and systems of the present disclosure. The mobile application may be used on or in conjunction with a mobile processing device, such as a tablet. In some embodiments, a computer program includes a mobile application provided to a mobile processor. In some embodiments, the mobile application is provided to a mobile processor at the time it is manufactured. In other embodiments, the mobile application is provided to a mobile processor via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, JavaScript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nintendo® DSi Shop.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location. The systems and methods disclosed herein may be implemented in the form of a mobile application or a computer software program.

In some embodiments, the platforms, systems, media, and methods disclosed herein include one or more databases, or use of the same. In view of the disclosure provided herein, those of skill in the art will recognize that many databases are suitable for storage and retrieval of raw image data, reconstructed image data, ROIs, stored classified data, label or classification, features, subcategory of features, etc. In various embodiments, suitable databases include, by way of non-limiting examples, relational databases, non-relational databases, object-oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices.

### Ultrasound System

Embodiments, variations, and examples of the methods and systems disclosed herein may be used in combination with an ultrasound system. For example, the ultrasound system may generate images, such as ultrasound tomography images, which may be used to generate a plurality of parameters as disclosed herein. An ultrasound system may be local or remote to the systems and methods for aiding a user to classify a volume of tissue disclosed herein. An ultrasound system may comprise an ultrasound tomography scanner. An ultrasound tomography scanner may comprise a transducer configured to receive the volume of tissue and comprising an array of ultrasound transmitters and an array of ultrasound receivers. The array of ultrasound transmitters may be configured to emit acoustic waveforms toward the volume of tissue, and the array of ultrasound receivers may be configured to detect a set of acoustic signals derived from acoustic waveforms transmitted through the volume of tissue. The ultrasound tomography scanner may further comprise a computer (e.g. an example of a digital processing device) in communication with the transducer, comprising one or more processors and non-transitory computer-readable media with instructions stored thereon that when executed may be configured to perform the methods of the present disclosures and embodiments and variations thereof described herein. The ultrasound tomography scanner may further comprise a display in communication with the digital processing device and configured to render the enhanced image of the volume of tissue.

The system may function to render ultrasound images and/or generate transformed ultrasound data that may be used to generate a high-resolution image of structures present within a volume of tissue. In some embodiments, the system may function to produce images that may be aligned with regulatory standards for medical imaging, as regulated, for instance, by the U.S. Food and Drug Administration (FDA). The system may be configured to implement at least a portion of an embodiment, variation, or example of methods described herein; however, the system may additionally or alternatively be configured to implement any other suitable method.

The transducer, the computer processor, and the display may be coupled to a scanner table. The scanner table may have an opening that provides access to the volume of tissue of the patient. The table, which may be made of a durable, flexible material (e.g., flexible membrane, fabric, etc.), may contour to the patient's body, thereby increasing scanning access to the axilla regions of the breast and increasing patient comfort. The opening in the table may allow the breast (or other appendage) to protrude through the table and be submerged in an imaging tank filled with water or another suitable fluid as an acoustic coupling medium that propagates acoustic waves.

A ring-shaped transducer with transducer elements may be located within the imaging tank and encircle or otherwise surround the breast, wherein each of the transducer elements may comprise one of the array of ultrasound transmitters paired with one of the array of ultrasound receivers. Multiple ultrasound transmitters that direct safe, non-ionizing ultrasound pulses toward the tissue and multiple ultrasound receivers that receive and record acoustic signals scattering from the tissue and/or transmitted through the tissue may be distributed around the ring transducer. In one embodiment, transducer may be organized such that each ultrasound transmitter element may be paired with a corresponding ultrasound receiver element, each ultrasound transmitter element may be surrounded by two adjacent ultrasound transmitter elements, each ultrasound receiver element may be surrounded by two adjacent ultrasound receiver elements, and the transducer may be axially symmetric.

During the scan, the ring transducer may pass along the tissue, such as in an anterior-posterior direction between the chest wall and the nipple region of the breast to acquire an acoustic data set including measurements such as acoustic reflection, acoustic attenuation, and sound speed. The data set may be acquired at discrete scanning steps, or coronal “slices”. The transducer may be configured to scan step-wise in increments from the chest wall towards the nipple, and/or from the nipple towards the chest wall. However, the transducer may additionally and/or alternatively receive data regarding any suitable biomechanical property of the tissue during the scan, and in any suitable direction.

In some embodiments, the scanner table may comprise an embodiment, variation, or example of the patient interface system described in any of the references incorporated herein and additionally or alternatively in U.S. application Ser. No. 14/208,181, entitled “Patient Interface System,” U.S. application Ser. No. 14/811,316 entitled “System for Providing Scanning Medium,” or P.C.T. International Pat. App. Pub. No. WO2017139389 entitled “System for Shaping and Positioning a Tissue Body,” which are each hereby incorporated by reference in their entirety. However, system 100 may additionally or alternatively comprise or be coupled with any other suitable patient interface system.

### Examples

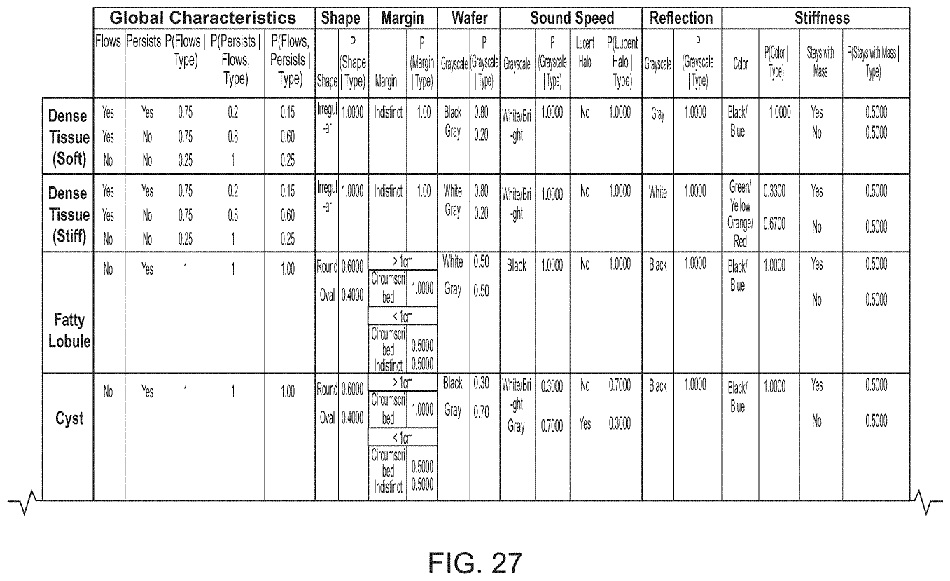

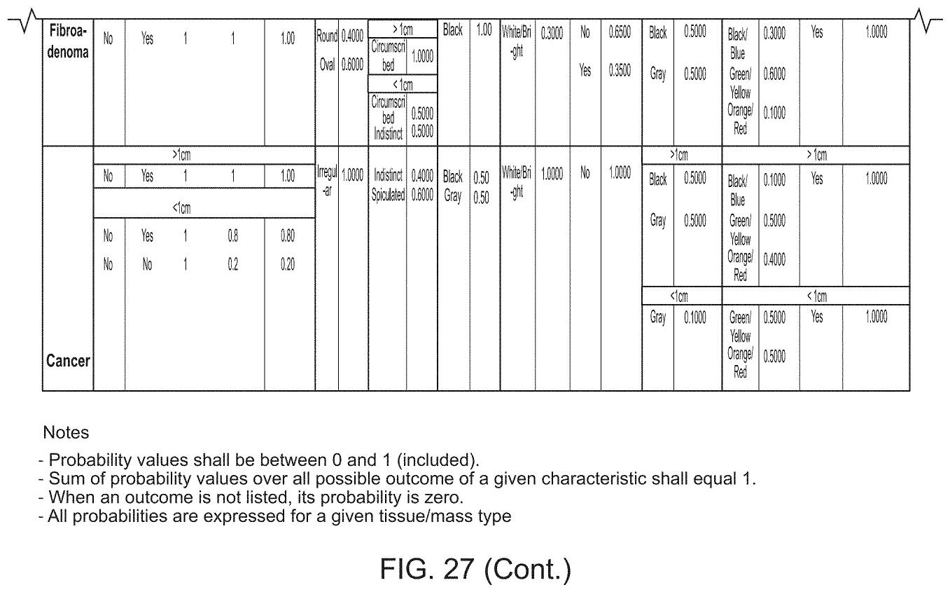

FIG. 27 is an example table of probabilities for associated parameter values and characterizations.

Table 2 shows an example characterization table.

TABLE 2
Characterization Table

| | | | | | | Sound | | | Scaling |
|---|---|---|---|---|---|---|---|---|---|
| | Flows | Persists | Shape | Margin | Wafer | Speed | Reflection | Stiffness | Factor |
| Dense Tissue | Yes | Yes | N/A | N/A | N/A | N/A | White | Black/Blue | 10.00 |
| (Soft) | | No | | | | | Gray | | |
| Dense Tissue | Yes | Yes | N/A | N/A | N/A | N/A | White | Green/Yellow | 10.00 |
| (Stiff) | | No | | | | | Gray | Orange/Red | |
| Fatty | No | Yes | N/A | N/A | N/A | Black | N/A | N/A | 10.00 |
| Lobule | | | | | | | | | |
| Not Fatty | No | Yes | N/A | N/A | N/A | White/Bright | N/A | N/A | 0.10 |
| Lobule | | | | | | Gray | | | |
| Cyst | No | Yes | N/A | Circumscribed | N/A | Gray | Black | Black/Blue | 10.00 |
| Fibroadenoma | No | Yes | Oval | Circumscribed | N/A | White/Bright | Black | Black/Blue | 10.00 |
| Cancer | No | Yes | N/A | Spiculated | N/A | N/A | N/A | N/A | 10.00 |
| | | No | | | | | | | |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A system for aiding a user to classify a volume of tissue, the system comprising:
a computer memory configured to store (i) a plurality of parameters associated with image characteristics related to one or more images of a volume of tissue; and (ii) a plurality of probabilities each associated with a potential classification of a region of the volume of tissue and each related to one or more parameters of the plurality of parameters, wherein the plurality of probabilities is assumed to be independent of one another; and
a graphical display visible to a user, the display comprising a graphical representation of a subset of relevant parameters of the plurality of parameters, wherein the graphical representation informs a classification of the region of the volume of tissue, and wherein a probability of the classification is represented visually.

2. The system of claim 1, wherein the graphical representation comprises a matrix style display wherein rows or columns of the matrix comprise all or a subset of the plurality of potential classifications of the image and wherein columns or rows comprise the subset of relevant parameters of the plurality of parameters, and wherein an element of the matrix provides a visible representation of a probability of a potential classification associated with a parameter of the subset of relevant parameters.

3. The system of claim 1, wherein the graphical representation comprises a parameter selection panel, wherein the parameter selection panel comprises all or a subset of the plurality of parameters.

4. The system of claim 3, wherein the parameter selection panel is visible on a user interface of an electronic device.

5. The system of claim 4, wherein the electronic device is a tablet or smartphone.

6. The system of claim 1, wherein a probability of the potential classification is displayed using a score value.

7. The system of claim 1, wherein a probability of the potential classification is displayed using a color saturation or grey scale variation.

8. The system of claim 1, wherein a probability of the potential classification is displayed using a size variation of a visual marker.

9. The system of claim 1, wherein a probability associated with the potential classification is a conditional probability.

10. The system of claim 9, wherein the conditional probability is computed using Bayes theorem.

11. The system of claim 1, wherein the image comprises one or more acoustic renderings of the volume of tissue, the one or more acoustic renderings comprising tissue characteristics related to sound propagation through the volume of tissue.

12. The system of claim 11, wherein the one or more acoustic renderings comprises at least a transmission image and a reflection image.

13. The system of claim 11, wherein the image comprises a combined or a derived image.

14. The system of claim 13, wherein the combined image comprises a plurality of reflection images.

15. The system of claim 13, wherein the combined image comprises a plurality of transmission images.

16. The system of claim 13, wherein the combined image comprises at least one reflection image and at least one transmission image.

17. The system of claim 11, wherein a transmission image comprises a sound speed image or an attenuation image.

18. The system of claim 1, wherein the region of the volume of tissue comprises a tissue lesion.

19. The system of claim 18, wherein the classification of the lesion comprises a cancer, a fibroadenoma, a cyst, a nonspecific benign mass, or an unidentifiable mass.

20. The system of claim 18, wherein the rows or the columns of the matrix represent the lesion type.

21. The system of claim 1, wherein the at least one image comprises at least one image selected from the group consisting of an enhanced reflection image, a B-mode reflection image, a sound speed image, and a stiffness representation.

22. The system of claim 1, wherein the plurality of parameters comprises at least one morphological feature.

23. The system of claim 1, wherein the plurality of parameters comprises at least one qualitative parameter.

24. The system of claim 1, wherein the plurality of parameters comprises at least one quantitative parameter.

25. The system of claim 1, wherein the plurality of parameters comprises at least one volumetric parameter.

26. The system of claim 1, wherein the plurality of parameters comprises at least one parameter derived from a plurality of image positions along an anterior-posterior axis of the tissue.

27. The system of claim 26, wherein the at least one parameter derived from the plurality of image positions comprises a comparison of a region margin or a region shape.

28. The system of claim 1, wherein the plurality of parameters comprises at least one parameter derived from two or more images selected from the group consisting of an enhanced reflection image, a B-mode reflection image, a sound speed image, and a stiffness representation.

29. The system of claim 28, the at least one parameter derived from two or more image types comprises a comparison of a region margin or a region shape.

30. The system of claim 1, wherein the graphical representation provides classification guidance.

31. The system of claim 1, wherein the system for aiding a user to classify a volume of tissue does not provide a classification to the user.

32. A computer-implemented method for aiding a user to classify an image of a volume of tissue, the method comprising:
receiving at a processor a set of parameters associated with image characteristics related to at least one image of the volume of tissue;
providing from the processor a set of probabilities associated with a potential classification of a region of the volume of tissue and each related to at least one parameter of the set of parameters, wherein the set of probabilities are assumed to be independent of one another; and
using the processor to provide a graphical representation of the set of probabilities on a display visible to a user, wherein the graphical representation on the display comprises and indication of a subset of relevant parameters of the plurality of parameters to the user, wherein the graphical representation is configured to inform a classification of the region of the volume of tissue, and wherein a probability of the classification is represented visually.

33. The method of claim 32, wherein the graphical representation comprises a matrix style display wherein rows or columns of the matrix comprises all or a subset of the plurality of potential classifications of the image and wherein columns or rows comprise the subset of relevant parameters of the plurality of parameters, and wherein an element of the matrix provides a visible representation of a probability of a potential classification associated with a parameter of the subset of relevant parameters.

34. The method of claim 32, further comprising selecting one or more parameters from a parameter selection panel, wherein the parameter selection panel comprises all or a subset of the plurality of parameters.

35. The method of claim 34, wherein the parameter selection panel is visible on a user interface of an electronic device.

36. The method of claim 35, wherein the electronic device is a tablet or smartphone.

37. The method of claim 32, further comprising displaying a score value associated with a probability of the potential classification.

38. The method of claim 32, further comprising varying a color saturation or grey scale.

39. The method of claim 32, further comprising varying a size of a visual marker.

40. The method of claim 32, wherein a probability associated with the potential classification is a conditional probability.

41. The method of claim 40, wherein the conditional probability is computed using Bayes theorem.

42. The method of claim 32, wherein the image comprises one or more acoustic renderings of the volume of tissue, the one or more acoustic renderings comprising a tissue characteristic related to sound propagation through the volume of tissue.

43. The method of claim 42, wherein the one or more acoustic renderings comprises at least a transmission image and a reflection image.

44. The method of claim 42, further comprising combining two or more images to form a combined or a derived image.

45. The method of claim 44, wherein the combined image comprises a plurality of reflection images.

46. The method of claim 44, wherein the combined image comprises a plurality of transmission images.

47. The method of claim 44, wherein the combined image comprises at least one reflection image and at least one transmission image.

48. The method of claim 42, wherein a transmission image comprises a sound speed image or an attenuation image.

49. The method of claim 32, wherein the region of the volume of tissue comprises a tissue lesion.

50. The method of claim 49, wherein the classification of the lesion comprises a cancer, a fibroadenoma, a cyst, a nonspecific benign mass, or an unidentifiable mass.

51. The method of claim 49, wherein the rows or the columns of the matrix represent the lesion type.

52. The method of claim 32, wherein the at least one image comprises at least one image selected from the group consisting of an enhanced reflection image, a B-mode reflection image, a sound speed image, and a stiffness representation.

53. The method of claim 32, wherein the plurality of parameters comprises at least one morphological feature.

54. The method of claim 32, wherein the plurality of parameters comprises at least one qualitative parameter.

55. The method of claim 32, wherein the plurality of parameters comprises at least one quantitative parameter.

56. The method of claim 32, wherein the plurality of parameters comprises at least one volumetric parameter.

57. The method of claim 32, wherein the plurality of parameters comprises at least one parameter derived from a plurality of image positions along an anterior-posterior axis of the tissue.

58. The method of claim 57, wherein the at least one parameter derived from the plurality of image positions comprises a comparison of a region margin or a region shape.

59. The method of claim 32, wherein the plurality of parameters comprises at least one parameter derived from two or more of images selected from the group consisting of an enhanced reflection image, a B-mode reflection image, a sound speed image, and a stiffness representation.

60. The method of claim 59, the at least one parameter derived from two or more image types comprises a comparison of a region margin or a region shape.

61. The method of claim 32, wherein the method further comprises providing classification guidance.

62. The method of claim 32, wherein the method does not provide a classification to the user.

63. A computer-implemented method of training a user to classify an image, comprising the method of claim 32 and further comprising:
receiving a classification from the user; and
providing a known classification to the user.

64. A computer-implemented method of building a dataset comprising:
providing a set of classified image data;
providing a set of parameters of the set of classified image data, wherein the set of parameters are based on a particular image characteristic;
calculating a Bayes probability of each parameter of the set of parameters yielding a characterization; and
storing a set of Bayes probabilities based on the Bayes probability of each parameter of the set of parameters in a database.

65. The method of claim 64, further comprising performing the method for aiding a user to classify a volume of tissue.

66. The method of claim 65, further comprising updating the database based on the classification of the region of the volume of tissue.

67. A non-transitory computer readable medium comprising machine-executable code that upon execution by a computing system implements the method of claim 32 or 64.

68. A system for aiding a user to classify a volume of tissue, the system comprising:
a computing system comprising a memory, the memory comprising instructions aiding a user to classify a volume of tissue, wherein the instructions when executed by a processor are configured to at least:
receive a set of parameters associated with image characteristics related to at least one image of the volume of tissue;
provide a set of probabilities associated with a potential classification of a region of the volume of tissue and each related to at least one parameter of the set of parameters, wherein the set of probabilities are assumed to be independent of one another; and
graphically represent the set of probabilities on a display visible to a user, wherein a graphical representation on the display indicates a subset of relevant parameters of the plurality of parameters to the user, wherein the graphical representation informs a classification of the region of the volume of tissue, and wherein a probability of the classification is represented visually.

69. The system of claim 68, wherein the graphical representation comprises a matrix style display wherein rows or columns of the matrix comprise all or a subset of the plurality of potential classifications of the image and wherein columns or rows comprise the subset of relevant parameters of the plurality of parameters, and wherein an element of the matrix provides a visible representation of a probability of a potential classification associated with a parameter of the subset of relevant parameters.

70. The system of claim 68, further comprising a parameter selection panel, wherein the parameter selection panel comprises all or a subset of the plurality of parameters.

71. The system of claim 70, wherein the parameter selection panel is visible on a user interface of an electronic device.

72. The system of claim 71, wherein the electronic device is a tablet or smartphone.

73. The system of claim 68, wherein a probability of the potential classification is displayed using a score value.

74. The system of claim 68, wherein a probability of the potential classification is displayed using a color saturation or grey scale variation.

75. The system of claim 68, wherein a probability of the potential classification is displayed using a size variation of a visual marker.

76. The system of claim 68, wherein a probability associated with the potential classification is a conditional probability.

77. The system of claim 76, wherein the conditional probability is computed using Bayes theorem.

78. The system of claim 68, wherein the image comprises one or more acoustic renderings of the volume of tissue, the one or more acoustic renderings comprising tissue characteristics related to sound propagation through the volume of tissue.

79. The system of claim 78, wherein the one or more acoustic renderings comprises at least a transmission image and a reflection image.

80. The system of claim 78, wherein the image comprises a combined or a derived image.

81. The system of claim 80, wherein the combined image comprises a plurality of reflection images.

82. The system of claim 80, wherein the combined image comprises a plurality of transmission images.

83. The system of claim 80, wherein the combined image comprises at least one reflection image and at least one transmission image.

84. The system of claim 78, wherein a transmission image comprises a sound speed image or an attenuation image.

85. The system of claim 68, wherein the region of the volume of tissue comprises a tissue lesion.

86. The system of claim 85, wherein the classification of the lesion comprises a cancer, a fibroadenoma, a cyst, a nonspecific benign mass, or an unidentifiable mass.

87. The system of claim 85, wherein the rows or the columns of the matrix represent the lesion type.

88. The system of claim 68, wherein the at least one image comprises at least one image selected from the group consisting of an enhanced reflection image, a B-mode reflection image, a sound speed image, and a stiffness representation.

89. The system of claim 68, wherein the plurality of parameters comprises at least one morphological feature.

90. The system of claim 68, wherein the plurality of parameters comprises at least one qualitative parameter.

91. The system of claim 68, wherein the plurality of parameters comprises at least one quantitative parameter.

92. The system of claim 68, wherein the plurality of parameters comprises at least one volumetric parameter.

93. The system of claim 68, wherein the plurality of parameters comprises at least one parameter derived from a plurality of image positions along an anterior-posterior axis of the tissue.

94. The system of claim 93, wherein the at least one parameter derived from the plurality of image positions comprises a comparison of a region margin or a region shape.

95. The system of claim 68, wherein the plurality of parameters comprises at least one parameter derived from two or more images selected from the group consisting of an enhanced reflection image, a B-mode reflection image, a sound speed image, and a stiffness representation.

96. The system of claim 95, the at least one parameter derived from two or more image types comprises a comparison of a region margin or a region shape.

97. The system of claim 68, wherein the graphical representation provides classification guidance.

98. The system of claim 68, wherein the system for aiding a user to classify a volume of tissue does not provide a classification to the user.

99. A method of classifying a lesion within a volume of tissue, the method comprising:
receiving from an ultrasound transducer at least one reflection rendering comprising sound reflection data within the volume of tissue;
identifying a region of interest within the at least one reflection rendering;
receiving from the ultrasound transducer at least one combined rendering comprising sound speed data and sound reflection data within the volume of tissue;
identifying a second region of interest within the at least one combined rendering; and
classifying a lesion within the volume of tissue based on a similarity or lack thereof of the first region of interest and the second region of interest.

100. The method of claim 99, further comprising determining a qualitative image parameter based on a similarity or lack thereof of the first region of interest and the second region of interest.

101. The method of claim 100, further comprising inputting the qualitative image parameter into a classifier model.

102. The method of claim 100, further comprising inputting the qualitative image parameter into the method for aiding a user to classify the volume of tissue.

103. A method of classifying a lesion within a volume of tissue, the method comprising:
receiving from an ultrasound transducer a first speed rendering at a first anterior-posterior position, the first speed rendering comprising sound speed data within the volume of tissue;
identifying a region of interest within the first speed rendering;
receiving from the ultrasound transducer a second speed rendering at a second anterior-posterior position, the second speed rendering comprising sound speed data within the volume of tissue;
identifying a second region of interest within the second speed rendering; and
classifying a lesion within the volume of tissue based on a similarity or lack thereof of the first region of interest and the second region of interest.

104. The method of claim 103, further comprising determining a qualitative image parameter based on a similarity or lack thereof of the first region of interest and the second region of interest.

105. The method of claim 104, further comprising inputting the qualitative image parameter into a classifier model.

106. The method of claim 104, further comprising inputting the qualitative image parameter into the method for aiding a user to classify the volume of tissue of claim 32 or 64.

107. A method of classifying a lesion within a volume of tissue, the method comprising:
receiving from an ultrasound transducer a first stiffness rendering at a first anterior-posterior position, the first stiffness rendering comprising a combination of sound speed and sound attenuation data within the volume of tissue;
identifying a region of interest within the first attenuation rendering;
receiving from the ultrasound transducer a second attenuation rendering at a second anterior-posterior position, the second attenuation rendering comprising a second combination of sound speed and sound attenuation within the volume of tissue;
identifying a second region of interest within the second attenuation rendering; and
classifying a lesion within the volume of tissue based on a similarity or lack thereof of the first region of interest and the second region of interest.

108. The method of claim 107, further comprising determining a qualitative image parameter based on a similarity or lack thereof of the first region of interest and the second region of interest.

109. The method of claim 108, further comprising inputting the qualitative image parameter into a classifier model.

110. The method of claim 108, further comprising inputting the qualitative image parameter into the method for aiding a user to classify the volume of tissue of claim 32 or 64.
